SEQUENCE LISTING (23 sequences)

SEQ ID NO: 1 - protein, 4 aa, Artificial Sequence
  Synthetic Peptide
YXXQ

SEQ ID NO: 2 - protein, 4 aa, Artificial Sequence
  Synthetic Peptide
XXXQ

SEQ ID NO: 3 - protein, 12 aa, Artificial Sequence
  Synthetic Peptide
LPVPEXINQSVP

SEQ ID NO: 4 - protein, 12 aa, Artificial Sequence
  Synthetic Peptide
VQNPVXHNQPLN

SEQ ID NO: 5 - protein, 12 aa, Artificial Sequence
  Synthetic Peptide
TDSNFXRALMDE

SEQ ID NO: 6 - protein, 12 aa, Artificial Sequence
  Synthetic Peptide
LPVPEXINRSVP

SEQ ID NO: 7 - protein, 12 aa, Artificial Sequence
  Synthetic Peptide
LPVPEXINESVP

SEQ ID NO: 8 - protein, 12 aa, Artificial Sequence
  Synthetic Peptide
LPVPEXINMSVP

SEQ ID NO: 9 - protein, 12 aa, Artificial Sequence
  Synthetic Peptide
LPVPEXINLSVP

SEQ ID NO: 10 - protein, 12 aa, Artificial Sequence
  Synthetic Peptide
LPVPEYINQSVP

SEQ ID NO: 11 - protein, 12 aa, Artificial Sequence
  Synthetic Peptide
VQNPVYHNQPLN

SEQ ID NO: 12 - protein, 12 aa, Artificial Sequence
  Synthetic Peptide
VGNPEXLNTVQP

SEQ ID NO: 13 - protein, 12 aa, Artificial Sequence
  Synthetic Peptide
LDNPDXQQDFFP

SEQ ID NO: 14 - protein, 769 aa, Artificial Sequence
  Synthetic Peptide
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTPTTCSNTIDLPMSPRTLDSLMQFGNNGEGAEPSAGGQFESLTFDMELTSECATSPM

SEQ ID NO: 15 - protein, 1210 aa, Artificial Sequence [sequence omitted]

SEQ ID NO: 16 - protein, 836 aa, Artificial Sequence
  Synthetic Peptide
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 17 - protein, 12 aa, Artificial Sequence
  Synthetic Peptide
TLVQTXVLQGDP

SEQ ID NO: 18 - protein, 12 aa, Artificial Sequence
  Synthetic Peptide
TLVQTYVLQGDP

SEQ ID NO: 19 - protein, 12 aa, Artificial Sequence
  Synthetic Peptide
PGPGHXLRCDST

SEQ ID NO: 20 - protein, 12 aa, Artificial Sequence
  Synthetic Peptide
PGPGHYLRCDST

SEQ ID NO: 21 - protein, 12 aa, Artificial Sequence
  Synthetic Peptide
SDQVLXGQLLGS

SEQ ID NO: 22 - protein, 12 aa, Artificial Sequence
  Synthetic Peptide
PSPLSXENLTFQ

SEQ ID NO: 23 - protein, 4 aa, Artificial Sequence
  Synthetic Peptide
XXXQ

(19) United States
(12) Patent Application Publication
(10) Pub. No.: US 2006/0148715 A1
(43) Pub. Date: Jul. 6, 2006

(54) Structural requirements for STAT3 binding and recruitment to phosphotyrosine ligands

(75) Inventor: David J. Tweardy, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 11/313,104

(22) Filed: Dec. 20, 2005

Related U.S. Application Data
(60) Provisional application No. 60/637,489, filed on Dec. 20, 2004.

(51) Int. Cl. A61K 38/10

(52) U.S. Cl. 514/14

(57) ABSTRACT

Inhibitors of Stat3 are disclosed, including small molecules and peptide mimetic inhibitors. Specific Stat3 inhibitors of the invention are useful as beta-turn mimetics. Also disclosed are pharmaceutical compositions of the Stat3 inhibitors of the invention, and methods for using the compounds of the invention to inhibit growth of a cell or to inhibit protein-protein interactions modulated by SH2 domains. Methods of screening for Stat3 inhibitors are also provided.

The present application claims priority to U.S. Provisional Patent Application Ser. No. 60/637,489, filed Dec. 20, 2004, which is incorporated by reference herein in its entirety.

### STATEMENT REGARDING FEDERALLY SPONSORED RESEARCH OR DEVELOPMENT

The present invention was developed with funds from the United States Government grant number CA86430. Therefore, the United States Government may have certain rights in the invention.

### FIELD OF THE INVENTION

The present invention is related to the field of molecular biology, structural biology, cell biology, and medicine. The present invention is also related to the field of signal transduction and inhibitors of signal transduction. The invention is directed towards inhibitors of Stat3, peptide mimetic inhibitors of Stat3, and methods to treat disease using Stat3 inhibitors.

### BACKGROUND OF THE INVENTION

Signal transducer and activator of transcription (STAT) 3 is a latent transcription factor activated by cytokine and growth factor receptors including IL-6 and EGFR (Wegenka et al., 1993; Akira et al., 1994; Zhong et. al, 1994) and granulocyte colony-stimulating factor (G-CSF) (Tian et al, 1994; Tweardy et al., 1995; Chakraborty et al., 1996). Stat3 is recruited to the cytoplasmic domain of receptors via its SH2 domain and phosphorylated on tyrosine 705 by either intrinsic or receptor-associated tyrosine kinases, most notably members of the Janus (JAK) family. Phosphorylation of Stat3 leads to dimerization mediated by reciprocal SH2-pY705 motif interactions, followed by nuclear translocation, binding to specific DNA elements, and up-regulation of target genes.

Stat3 has been demonstrated to be required for transformation of fibroblasts by v-Src (Turkson et. al, 1998; Bromberg et. al, 1998) and for autocrine growth of squamous cell carcinoma of the head and neck (SCCHN) (Grandis et. al, 1998) where it is activated by an autocrine loop involving TGF-β and EGFR (Grandis et. al, 1993). Expression of a constitutively activated form of Stat3 alone in fibroblasts was oncogenic (Bromberg et. al, 1999). Constitutive activation of Stat3 occurs in a wide variety of cancers in addition to SCCHN including breast, prostate, renal cell, melanoma, ovarian, lung, leukemia, lymphoma, and multiple myeloma (Bowman et. al, 2000) as a result of autocrine or paracrine activation of the EGFR and the IL-6R or secondary to one or more as yet unidentified mechanisms.

EGFR contains an extracellular ligand-binding domain, a single transmembrane region and an intracellular domain harboring intrinsic tyrosine kinase activity (Ullrich et. al, 1984). Ligand-induced dimerization of EGFR allows reciprocal transphosphorylation of residues within the catalytic domain of the kinase leading to its enzymatic activation and autophosphorylation of C-terminal cytoplasmic tyrosine residues. Five autophosphorylation sites have been identified in EGFR—Y992, Y1068, Y1086, Y1148 and Y1173 (Downward et. al, 1984; Margolis et. al, 1990). These phosphorylated tyrosine residues serve as docking sites for signal proteins containing Src homology (SH2) domains, including phospholipase C-γ (Rotin et. al, 1984; Chattopadhyay et. al, 1999), Grb-2 (Okutani et. al, 1994; Batzer et. al, 1994), Shc (Okabayashi et. al, 1994) SHP-1 (Keihack et. al, 1998) and most recently Stat3 (Shao et. al, 2003), which was shown by the inventors to bind to EGFR at pY sites located at Y1068 and Y1086. Both of these tyrosine residues are followed at the pY +3 position by Q, thereby conforming to the consensus Stat3 SH2-binding motif, YXXQ (SEQ ID NO:1) (Stahl et. al, 1995; Weber-Nordt et. al, 1996).

The G-CSF receptor (G-CSFR) is a member of the type I cytokine receptor family. Ligand-induced dimerization of the G-CSFR results in activation of receptor-associated protein tyrosine kinases (PTK), most notably those of the Jak kinase family. Studies were performed to assess the physiological role of Stat3 activation by G-CSF in which wild type and dominant negative Stat3 constructs were overexpressed in myeloid cell lines and murine bone marrow progenitor cells. The studies supported the concept that the role of Stat3 in G-CSFR signaling in normal myeloid progenitor cells is to promote cell survival and to help direct myeloid maturation. Studies examining oncogenic signaling pathways active at a single cell level in acute myeloid leukemia (AML) demonstrated that Stat3 activation by G-CSF was associated with relapse following initial chemotherapy in the subset of AML cells containing Flt3 with an internal duplication.

Activation of receptor-associated PTK results in phosphorylation of tyrosines located within the C-terminal end of the cytoplasmic domain of the receptor (Y704, Y729, Y744 and Y764 in the human receptor; Y703, Y728, Y743 and Y763 in the murine receptor) and recruitment of SH2-containing proteins to these sites including Shc to Y764; SHP-2 to Y704 and Y764; PI3K to Y704; SOCS-3 to Y704 and Y729; Grb2 and the adapter protein, 3BP2, to Y764; and Stat3 to Y704 and Y744. Following its recruitment to Y704 and Y744, Stat3 is phosphorylated on tyrosine 705 by receptor-associated Jak kinase family members leading to dimerization mediated by reciprocal SH2-pY705 motif interactions, nuclear translocation and binding to specific DNA elements. G-CSFR Y704 is followed at the +3 position by the polar amino acid residue Q, thereby conforming to the consensus Stat3 SH2-binding motif, YxxQ. Among the group of SH2-containing proteins that bind pY motifs within the G-CSFR, with the exception of Grb2, the structural basis for their pY binding preferences is poorly understood.

The preference of Stat3 SH2 for pY peptide ligands containing Q (or the polar residues T or C) at the +3 position is unique among SH2 domains. The structural basis for this is unknown but could be exploited to inhibit Stat3 activation in cancer. While the structure of Stat3 SH2 bound to pY ligand has not been solved, the structure of Stat3β bound to DNA has (Becker et. al, 1998) encompassing the domains of Stat3β from residues 127 to 722 including the SH2 domain. The authors concluded that Stat3 SH2 shares structural features of other SH2 domains having a central, three-stranded anti-parallel β-pleated sheet (strands B, C and D) flanked by helix αA and strands βA and PG. However, since the electron density was not well defined for the SH2 domain and the pY705-containing phosphopeptide region, the structure obtained did not clarify the preference of Stat3 SH2 for binding to phosphopeptide ligands with pY +3 Q (or +3 T since T708 is located at the +3 position downstream of pY705). Two models have been proposed to explain this preference (Hemmann et. al, 1996; Chakraborty et. al, 1999); both assume an extended configuration for the pY peptide ligand and two pockets—one is a positively charged pocket that interacts with the pY residue, and the other is a hydrophilic pocket that interacts with the +3 Q—but neither model has been fully tested and verified.

### BRIEF SUMMARY OF THE INVENTION

The present invention concerns compositions and methods related to Stat3, such as for the treatment and/or prevention of cancer. In some embodiments, the compositions and methods concern treatment of chemotherapy-resistant cancer and/or prevention of the development thereof. Specific compositions may inhibit the binding of Stat3 to any other molecule and/or the activation (such as upon forming a particular structural configuration) of Stat3. Such compositions may affect the binding of Stat3 to receptor complexes or other Stat3-activating complexes, phosphorylation of Stat3, as well as the DNA-binding activity of Stat3, in particular aspects of the invention.

This invention is the first to demonstrate the structural basis for Stat3 SH2 domain binding to phosphotyrosine ligands, such that the amide hydrogen located at Stat3 residue E638 forms a bond with the oxygen molecule on the side chain of the +3Q residue. The present invention demonstrates that Stat3 recruitment and activation by the CSFR at the Y704 residue occurs through a critical interaction of the Stat3 R609 side chain with Y704, which is followed by or is concurrent with the receptor in the regions of this tyrosine forming a P turn, which facilitates the formation of a bond between the amide hydrogen of residue E638 with the oxygen on the +3Q residue of the receptor. The same model governs Stat3 binding to the EGFR, such that pY binding to the Stat3 SH2 domain requires interaction of the phosphate group on the tyrosine residue with Stat3 residues K589 and R609 and the formation of a bond between the amide hydrogen of Stat3 E638 with the oxygen on the side chain of the +3Q residue. The models proposed by Chakraborty and Hemmann ((Hemmann et al., 1996; Chakraborty et al., 1999), respectively, propose involvement of the side chains of E638, Y640, and Y657 or Y657, C687, S691 and Q692 (proposed to form pocket 2) of the Stat3 SH2 domain to facilitate binding to the receptor YXXQ motif. The invention disclosed herein demonstrates that Stat3 SH2 domain to YXXQ pY receptor ligands does not require the side chains proposed to form pocket 2 by the Chakraboty and Hemmann models.

In one embodiment of the invention, there is a Stat3 inhibitor comprising a beta-turn mimetic wherein said beta-turn mimetic is capable of binding to a sequence located within the SH₂ domain of Stat3. In a specific embodiment, the binding is with an affinity that is at least equal to the affinity of pY1068-epidermal growth factor receptor for Stat3. In an additional embodiment of the invention, the binding is with an affinity that is at least equal to the affinity of pY1086-epidermal growth factor receptor for Stat3. In another specific embodiment, the binding is with an affinity that is at least equal to the affinity of pY704-granulocyte colony-stimulating factor receptor for Stat3. In a further specific embodiment, the binding is with an affinity that is at least equal to the affinity of pY744-granulocyte colony-stimulating factor receptor for Stat3.

In particular aspects of the invention, the beta-turn mimetic has a low affinity for the SH₂ domain of Grb2. In additional aspects, the beta-turn mimetic is a mimetic of a beta-turn region comprising SEQ ID NO:2. The beta-turn mimetic may comprise a peptide, such as an amino-terminally modified peptide or a carboxy-terminally modified peptide. A peptide of the invention may comprise a combination of standard amino acids and modified amino acids, for example. Peptides of the invention may comprise the sequence of SEQ ID NO:2, SEQ ID NO:3 (pY1068 dodecapeptide), SEQ ID NO:4 (pY1086 dodecapeptide), SEQ ID NO:17 (pY704 dodecapeptide), SEQ ID NO:19 (pY744 dodecapeptide), or mixtures thereof, for example. In particular embodiments, X₂ of SEQ ID NO:2 is not asparagine.

In particular embodiments of a Stat3 inhibitor of the invention, the mimetic is cyclic. The mimetic may comprise a peptide having the exemplary sequence SEQ ID NO: 23 (X₁X₂X₃Q), wherein X₁ is a phosphotyrosine mimetic residue that is selected from the group consisting of phosphonomethylphenylalanine, difluorophosphonomethylphenylalanine, O-malonyltyrosine, and O-fluoromalonyltyrosine. In particular aspects, binding the sequence within the SH₂ domain may comprise interaction with residue E638 of Stat3 and/or the sequence within the SH₂ domain further comprises interaction with residues K589 and R607.

In another embodiment of the invention, there is a Stat3 inhibitor, wherein the inhibitor is capable of binding to the amide hydrogen of residue E638 of Stat3 with an affinity that is at least equal to the affinity of pY1068-epidermal growth factor receptor.

In an additional embodiment of the invention there is a Stat3 inhibitor, wherein the inhibitor is capable of binding to the amide hydrogen of residue E638 of Stat3 with an affinity that is at least equal to the affinity of pY1086-epidermal growth factor receptor.

In another embodiment of the invention there is a Stat3 inhibitor, wherein the inhibitor is capable of binding to the amide hydrogen of residue E638 of Stat3 with an affinity that is at least equal to the affinity of pY704-granulocyte colony stimulating factor receptor.

In a further embodiment there is a Stat3 inhibitor, wherein the inhibitor is capable of binding to the amide hydrogen of residue E638 of Stat3 with an affinity that is at least at least equal to the affinity of pY744-granulocyte colony stimulating factor receptor.

In an additional embodiment there is a pharmaceutical composition comprising any Stat3 inhibitor of the invention, and methods are contemplated of inhibiting Stat3 comprising administering to a mammal a Stat3 inhibitor of the invention. In particular embodiments, there are methods of treating cancer comprising administering to a mammal a Stat3 inhibitor of the invention. The cancer may be of any kind, but in particular embodiments the cancer is selected from the group consisting of head and neck, breast, prostate, renal cell, melanoma, ovarian, lung, leukemia, lymphoma, and multiple myeloma.

In a further embodiment of the present invention, there is a composition of FIG. 11, FIG. 12, or a mixture thereof. The composition may be further defined as a pharmaceutical composition, such as one comprised in a pharmaceutically acceptable excipient. In particular embodiments, one or more of these compositions are employed in methods to inhibit Stat3, methods to inhibit proliferation of a cell, such as a cancer cell, and/or methods to treat cancer.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows that Q at the +3 position within Y1068 peptide is required for Stat3 SH2 binding of peptide; The proteins were separated by SDS-PAGE and immunoblotted using Stat3 mAb (bottom panel).

FIGS. 2A-2D show models of Stat3 SH2-phosphotyrosine binding and Stat3 proteins generated to test them. FIG. 2A and FIG. 2B show prior art schematic representations of the two models of Stat3 SH2 binding to pYXXQ (SEQ ID NO:2) peptide proposed by Chakraborty (FIG. 2A) and Hemmann (FIG. 2B) each involving two pockets. In these models, the phosphotyrosine (pY) interacts with a positively-charged pocket formed by the side chains of K591, R609, S611, E612, and S613 (FIG. 2A) or by R609 (FIG. 2B) while pY +3 Q interacts with a hydrophilic pocket within the SH2 domain formed by the side chains of E638, Y640 and Y657 (FIG. 2A) or Y657, C687, S691 and Q692 (FIG. 1B). FIG. 2C shows mutations that were introduced at the amino acid residues indicated (+) to generate a panel of wild type and mutant Stat3 proteins. FIG. 2D shows wild type and mutant Stat3 proteins;

FIGS. 3A-3C show the requirement for R609 and K591, but not any of the proposed pocket 2 residues, for Stat3 SH2 binding to Y1068 and Y1086 PDP. FIG. 3A shows wild type or mutant Stat3 proteins mixed with the peptides shown in Table 2. FIG. 3B and FIG. 3C shows a mirror resonance affinity assay;

FIGS. 4A-4B shows a revised model of Stat3 SH2 binding to +3 Q within YXXQ-containing phosphopeptide ligands. FIG. 4A shows that computational modeling using the Biopolymer program in the Insight II environment was used to perform local energy optimization of the interaction of Stat3 SH2 (shown as a gray ribbon) with phosphopeptide ligand (EpYINQ shown as a green ribbon) based upon the known structures of each. FIG. 4B shows an overlay of the known structure of wild type Stat3 (green) with the predicted structure of Stat3-E638P (gray). The positions of the side chains of relevant residues are indicated for wild-type Stat3 (aqua stick models) and for Stat3E638 (gray stick models); and

FIGS. 5A-5C show expression and CD of Stat3-E638P and the effect of E638P mutation on Stat3 binding to EGFR-based PDP. FIG. 5A shows SDS-PAGE of Stat3-E638P protein stained with Coomassie Blue (upper panel) or immunoblotted with Stat3 mAb (lower panel). FIG. 5B shows CD spectrum of wild type Stat3 (squares) and Stat3-E638P (triangles). FIG. 5C shows wild type or mutant Stat3 proteins mixed with the peptides shown in Table 2.

FIGS. 6A-6C show the requirement for the side chains of K591 and R609 and the peptide amide hydrogen of E638, but not the side chains of any of the proposed pocket 2 residues, for Stat3 SH2 binding to Y704 and Y744 phosphododecapeptides. FIG. 6A shows NeutrAvidin agarose was incubated with the indicated biotinylated peptides (see Table 4 for sequence) or no peptide (CON) as control, washed thoroughly and mixed with identical amounts of wild type or mutant Stat3 proteins as indicated. Bound proteins were separated by SDS-PAGE and immunoblotted using Stat3 mAb. Lane ST represents purified wild type Stat3 (0.6 μg) loaded directly onto the gel as positive control. Mirror resonance affinity assay. Cells of a biotin-coated cuvette pretreated with saturating amounts of NeutrAvidin were pretreated with biotinylated phosphopeptide based on Y704, (FIG. 6B) or biotinylated phosphopeptide based on Y744 FIG. 6C. Wild type or mutated Stat3 protein was added in the concentrations indicated and mirror resonance measurements recorded continuously for 10 min as shown.

FIGS. 7A-7C show the revised model of Stat3 SH2 binding to +3 Q/C within YxxQ/C-containing phosphopeptide ligands. FIG. 7A shows computational modeling using the Biopolymer program in the Insight II environment was used to perform local energy optimization of the interaction of Stat3 SH2 with phosphopeptide ligand EpYINQ (contained within the EGFR and demonstrated to recruit both Stat3 and Grb2) based upon the known structures of each. As indicated, the oxygen on the side chain of the pY +3 Q within the EpYINQ peptide is predicted to form a hydrogen (H) bond with the amide hydrogen at E638 and to make a major contribution to the binding energy. The positions are shown for the side chains of E638, Y640 and Y657 proposed by Chakraborty to form pocket 2 and for the side chain of W623 proposed to force a β turn in the peptide ligand. Models of Stat3 binding to Y704 phosphopentapeptide ligand (FIG. 7B) and to Y744 phosphopentapeptide ligand (FIG. 7B).

FIGS. 8A-8C show the requirement for the side chain of R609 and the amide hydrogen of E638 for Stat3 binding to the G-CSFR and Stat3 phosphorylation on Y705 in vivo. 293T cells were transfected with G-CSFR alone or co-transfected with G-CSFR and either wild type Stat3 cDNA construct, mutant Stat3 cDNA construct or empty eukaryotic expression vector (pcDNA3.1) vector as indicated. After 48 h incubation, the cells were stimulated with G-CSF (100 ng/ml) for 15 min as indicated and the cells lysed. (A) Cell lysates were immunopreciptated with anti-G-CSFR antibody and protein G agarose (Sigma) at 4° C. for 2 h. Immunoprecipitates were separated by SDS-PAGE and immunoblotted for pStat3, total Stat3 and G-CSFR as indicated (FIG. 8B) Equal amounts of lysates based on protein content were separated by SDS-PAGE and immunoblotted for pStat3, total Stat3 and G-CSFR as indicated. (FIG. 8C) Cell lysates were incubated with Ni-NTA agarose (lanes 1-8). In lane 9 and 10, equal amounts of purified Stat3 were mixed with lysates from cells transfected by G-CSFR vector only before incubation with Ni-NTA agarose. Affinity-purified proteins were separated by SDS-PAGE and immunoblotted for pStat3 and total Stat3 as indicated.

FIG. 9 shows fluorescence microscopy of HepG2 cells transiently transfected with CFP-Stat3, pre-treated as indicated, and incubated without or with IL-6.

FIG. 10 illustrates a virtual ligand screening procedure with an exemplary candidate.

FIG. 11 provides the structure of an exemplary compound of the invention.

FIG. 12 provides the structures of additional exemplary compounds of the invention.

### DETAILED DESCRIPTION OF THE INVENTION

As used herein, the use of the word “a” or “an” when used in conjunction with the term “comprising” in the sentences and/or the specification may mean “one,” but it is also consistent with the meaning of “one or more,” “at least one,” and “one or more than one.” As used herein “another” may mean at least a second or more. Still further, the terms “having”, “including”, “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method or composition described herein can be implemented with respect to any other method or composition described herein.

As used herein, “binding affinity” refers to the strength of an interaction between two entities, such as a protein-protein interaction. Binding affinity is sometimes referred to as the Kₐ, or association constant, which describes the likelihood of the two separate entities to be in the bound state. Generally, the association constant is determined by a variety of methods in which two separate entities are mixed together, the unbound portion is separated from the bound portion, and concentrations of unbound and bound are measured. One of skill in the art realizes that there are a variety of methods for measuring association constants. For example, the unbound and bound portions may be separated from one another through adsorption, precipitation, gel filtration, dialysis, or centrifugation, for example. The measurement of the concentrations of bound and unbound portions may be accomplished, for example, by measuring radioactivity or fluorescence, for example. In certain embodiments of the invention, the binding affinity of a Stat3 inhibitor for the SH2 domain of Stat3 is similar to or greater than the affinity of the pY-1068 or pY-1086-containing beta-turns of EGFR for the SH2 domain of Stat3. In other embodiments of the invention, the binding affinity of a Stat3 inhibitor for the SH2 domain of Stat3 is similar to or greater than the affinity of the pY-704 or pY-744-comprising beta-turns of G-CSFR for the SH2 domain of Stat3.

The term “chemotherapy-resistant cancer” as used herein refers to cancer that is suspected of being unable to be treated with one or more particular chemotherapies or that is known to be unable to be treated with one or more particular chemotherapies. In particular, cells of the chemotherapy-resistant cancer are not killed or rendered quiescent with the therapy or even continue to multiply during or soon after the therapy.

The term “domain” as used herein refers to a subsection of a polypeptide that possesses a unique structural and/or functional characteristic; typically, this characteristic is similar across diverse polypeptides. The subsection typically comprises contiguous amino acids, although it may also comprise amino acids that act in concert or that are in close proximity due to folding or other configurations. An example of a protein domain is the SH2 domain of Stat3. The term “SH2 domain” is art-recognized, and, as used herein, refers to a protein domain involved in protein-protein interactions, such as a domain of a Src tyrosine kinase that regulates kinase activity. The invention contemplates modulation of activity, such as activity dependent upon protein-protein interactions, mediated by SH2 domains of proteins (e.g., tyrosine kinases such as src) or proteins involved with transmission of a tyrosine kinase signal in organisms including mammals, such as humans.

The term “inhibitor” as used herein refers to one or more molecules that interfere at least in part with the activity of Stat3 to perform one or more activities, including with the ability of Stat3 to bind to a molecule and/or the ability to be phosphorylated.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, and chimpanzees. Mammals may be referred to as “patients”.

The language “modulating an activity mediated by an SH2 domain” as used herein, refers to inhibiting, abolishing, or increasing the activity of a cell-signaling pathway mediated by a protein including an SH2 domain, e.g., by disrupting protein-protein interactions mediated by SH2 domains. In a preferred embodiment, an activity mediated by an SH2 domain is inhibited, for example, an interaction of Stat3 and EGFR is inhibited. In another preferred embodiment, an interaction of Stat3 and G-CSFR is inhibited.

“Peptide” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, beta-alanine, phenylglycine and homoarginine are also included. The amino acids may be either the D- or L-isomer. The L-isomers are generally preferred. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

“Protein”, as used herein, means any protein, including, but not limited to peptides, polypeptides, enzymes, glycoproteins, hormones, receptors, antigens, antibodies, growth factors, etc., without limitation. Presently preferred proteins include those comprised of at least 25 amino acid residues, more preferably at least 35 amino acid residues and still more preferably at least 50 amino acid residues.

I. Peptide Mimetics and Synthesis Thereof

As used herein, the terms “mimetic” or “peptide mimetic” may be used interchangeably and refer to a compound that biologically mimics determinants on hormones, cytokines, enzyme substrates, viruses or other bio-molecules, and may antagonize, stimulate, or otherwise modulate the physiological activity of the natural ligands. Certain exemplary mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule. Molecules are designed to recognize amino acid residues in alpha-helix or beta-turn conformations on the surface of a protein. Such molecules may be used in disrupting certain protein-protein interactions involved in disease. In a preferred embodiment, mimetics of the present invention are inhibitors of Stat3. In a preferred embodiment, the inhibitors of Stat3 are “beta-turn mimetics.” In another preferred embodiment, the mimetics of the present invention mimic a beta-turn region of EGFR that interacts with Stat3. In another preferred embodiment, the mimetics of the present invention mimic a beta-turn region of G-CSFR that interacts with Stat3.

Peptide mimetics can be designed and produced by techniques known to those of skill in the art. (See e.g., U.S. Pat. Nos. 4,612,132; 5,643,873 and 5,654,276, the teachings of which are herein incorporated by reference). These mimetics can be based, for example, on one or more specific peptide phosphatase inhibitor sequences and maintain the relative positions in space of the corresponding peptide inhibitor. These peptide mimetics possess biologically activity (e.g., phosphatase inhibiting or stimulating activity) similar to the biological activity of the corresponding peptide compound, but possess a “biological advantage” over the corresponding peptide inhibitor or stimulation with respect to one, or more, of the following properties: solubility, stability, and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic inhibitor. Modifications of peptides to produce peptide mimetics are described in U.S. Pat. Nos. 5,643,873 and 5,654,276, the teachings of which are incorporated herein by reference.

Where the peptide mimetics of present invention comprise amino acids, the test substance can also be cyclic protein, peptides and cyclic peptide mimetics. Such cyclic test substances can be produced using known laboratory techniques (e.g., as described in U.S. Pat. No. 5,654,276, the teachings of which are herein incorporated in their entirety by reference).

The mimetics of the present invention can comprise either the 20 naturally occurring amino acids or other synthetic amino acids. Synthetic amino acids encompassed by the present invention include, for example, naphthylalanine, L-hydroxypropylglycine, L-3,4-dihydroxyphenylalanyl, alpha-amino acids such as L-alpha-hydroxylysyl and D-alpha-methylalanyl, L-alpha-methyl-alanyl, beta amino-acids such as beta-analine, and isoquinolyl, for example.

D-amino acids and other non-naturally occurring synthetic amino acids can also be incorporated into the test substances of the present invention. Such other non-naturally occurring synthetic amino acids include those where the naturally occurring side chains of the 20 genetically encoded amino acids (or any L or D amino acid) are replaced with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic.

As used herein, “lower alkyl” refers to straight and branched chain alkyl groups having from 1 to 6 carbon atoms, such as methyl, ethyl, propyl, butyl and so on. “Lower alkoxy” encompasses straight and branched chain alkoxy groups having from 1 to 6 carbon atoms, such as methoxy, ethoxy and so on.

Cyclic groups can be saturated or unsaturated, and if unsaturated, can be aromatic or non-aromatic. Heterocyclic groups typically contain one or more nitrogen, oxygen, and/or sulphur heteroatoms, e.g., furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. The heterocyclic groups can be substituted or unsubstituted. Where a group is substituted, the substituent can be alkyl, alkoxy, halogen, oxygen, or substituted or unsubstituted phenyl. (See U.S. Pat. No. 5,654,276 and U.S. Pat. No. 5,643,873, the teachings of which are herein incorporated by reference).

The peptide analogs or mimetics of the invention include isosteres. The term “isostere” as used herein refers to a sequence of two or more residues that can be substituted for a second sequence because the steric conformation of the first sequence fits a binding site specific for the second sequence. The term specifically includes peptide back-bone modifications (i.e., amide bond mimetics) well known to those skilled in the art. Such modifications include modifications of the amide nitrogen, the alpha-carbon, amide carbonyl, complete replacement of the amide bond, extensions, deletions or backbone crosslinks. Several peptide backbone modifications are known, including ψ[CH₂S], ψ[CH₂NH], ψ[C(S)NH₂], ψ[NHCO], ψ[C(O)CH₂], and ψ[(E) or (Z) CH═CH]. In the nomenclature used above, ψ indicates the absence of an amide bond. The structure that replaces the amide group is specified within the brackets. Other examples of isosteres include peptides substituted with one or more benzodiazepine molecules (see e.g., James, G. L. et al. (1993) Science 260:1937-1942).

Other possible modifications include an N-alkyl (or aryl) substitution (ψ[CONR]), backbone crosslinking to construct lactams and other cyclic structures, or retro-inverso amino acid incorporation (ψ[NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide and is able to bind the selected SH2 domain. See Goodman et al. “Perspectives in Peptide Chemistry” pp. 283-294 (1981). See also U.S. Pat. No. 4,522,752 by Sisto for further description of “retro-inverso” peptides.

II. Beta-Turns

Beta-turns are protein secondary structure elements that are commonly found to link two strands of anti-parallel beta-sheet, forming a beta-hairpin. Beta-turns are generally about 2-7 amino acids in length. In general, a beta-turn (or reverse turn, as it they are sometimes called) is any region of a protein where there is a hydrogen bond involving the carbonyl of residue i and the NH group of residue i+3. An alternative definition states that the alpha-carbons of residues i and i+3 must be within 7.0 Angstroms. In certain embodiments of the invention, the beta-turn is within a region of the protein EGFR. In a preferred embodiment, the beta-turn comprises the residue Y-1068 or Y-1086 of EGFR. In certain embodiments of the invention, the beta-turn is within a region of the protein G-CSFR, such as the region comprising Y-704 or Y-744.

III. Methods of Modulating Interactions Mediated by SH2 Domains

In still another aspect, the invention provides methods for modulating an activity mediated by an SH2 domain. In general, the methods include the step of contacting an SH2 domain with a compound of the invention, such that activity of the SH2 domain is modulated. In a preferred embodiment, the SH2 domain is the SH2 domain of Stat3. An example of a Stat3 protein contemplated in the present invention is SEQ ID No: 14.

The methods of the invention provide means for inhibiting protein-protein interactions mediated by SH2 domains. Proteins with SH2 domains couple protein-tyrosine kinases to signalling networks involved in growth regulation. Disruption of growth-regulatory signal transduction can result in inhibition of cell growth. Accordingly, the invention provides methods for inhibiting growth of cells, including microbial cells and transformed cells, e.g., by inhibiting protein-protein interactions mediated by SH2 domains involved in growth-regulatory signal transduction. Thus, the invention provides methods for treating conditions associated with abnormal or undesired cell growth, including, e.g., fungal or bacterial infections, neoplastic conditions (including cancer), and the like.

In one embodiment, the invention provides a method for modulating intracellular signaling pathways by disrupting particular protein-protein interactions mediated by SH2 domains. For instance, the SH2 inhibitors of the present invention can be used to affect the responsiveness of a cell to a growth factor, cytokine or other receptor ligand, and to inhibit the proliferation of transformed cells or to render transformed cells more sensitive to cytostatic or cytotoxic agents. The SH2 target of the subject inhibitors can range from the interaction between, for example, an activated receptor complex and the initial cytoplasmic proteins involved in triggering a particular set of intracellular signaling pathways, to the last SH2-mediated interaction in a specific pathway, such as the formation of a transcription factor complex or allosteric regulation of an enzymatic activity. Thus, the inhibitors of the present invention can be used to inhibit the interaction between an SH2-binding signal transduction protein such as EGFR, an example of an EGFR contemplated by the present invention is SEQ ID NO:15, and such SH2-containing proteins as, for example, phospholipase C-γ, Grb-2 Shc, Stat3, and SHP-1. The inhibitors of the present invention can be used to inhibit the interaction between an SH2-binding signal transduction protein such as G-CSFR; an example of an G-CSFR contemplated by the present invention is SEQ ID NO:16.

Interaction with SH2 domains can lead to activation of the biochemical function associated with the target protein. In a preferred embodiment, the methods of the invention for inhibition of protein-protein interactions mediated by SH2 domains include the step of contacting an SH2 domain with a Stat3 inhibitor of the present invention. In preferred embodiments, the compound is selected to preferentially inhibit an SH2 domain of an abnormal cell (such as a cancer cell), or a pathogen cell (e.g., a fungal pathogen). Thus, in preferred embodiments, the methods of the invention comprise contacting an SH2 domain of a target protein with a compound of the invention that is selective for the target protein SH2 domain.

Stat3 inhibitors useful in the methods of the invention can be determined by the skilled artisan in light of the teaching herein using no more than routine experimentation. Described herein are methods of determining the binding of various peptides to the SH2 region of Stat3. Other assays that measure the ability of a compound to inhibit proliferation, to alter the responsiveness of a cell to a growth factor, and the like, will be apparent to the ordinarily-skilled artisan. For example, the ability of a compound of the invention to inhibit cell growth in culture can be measured by standard assays.

IV. Proteinaceous Compositions

In certain embodiments, the present invention concerns at least one proteinaceous molecule. As used herein, a “proteinaceous molecule,” “proteinaceous composition,” “proteinaceous compound,” “proteinaceous chain” or “proteinaceous material” generally refers, but is not limited to, a protein or polypeptide of at least two amino acids. All the “proteinaceous” terms described above may be used interchangeably herein.

In certain embodiments the size of the at least one proteinaceous molecule may comprise, but is not limited to, a molecule having about 2 to about 2500 or greater amino molecule residues, and any range derivable therein. The invention includes those lengths of contiguous amino acids of any sequence discussed herein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

In certain embodiments, the proteinaceous composition comprises at least one protein, polypeptide or peptide. In methods that involve an inhibitor of Stat3 polypeptide, the inhibitor may comprise a protein, and as such, a composition comprising the inhibitor is a proteinacious composition of the present invention. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide comprising compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides, or peptides through standard molecular biological techniques, the isolation of proteinaceous compounds from natural sources, or the chemical synthesis of proteinaceous materials, for example. The nucleotide, protein, polypeptide, and peptide sequences for various polynucleotides (such as genes, for example) have been previously disclosed, and may be found at computerized databases known to those of ordinary skill in the art. One such database is the National Center for Biotechnology Information's Genbank® and GenPept® databases. The coding regions for these known genes may be amplified and/or expressed using the techniques disclosed herein or as would be known to those of ordinary skill in the art. Alternatively, various commercial preparations of proteins, polypeptides, and peptides are known to those of skill in the art.

In certain embodiments, a proteinaceous compound may be purified. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide-comprising component may be used in the compositions and methods disclosed herein. However, it is preferred that the proteinaceous material is biocompatible. In certain embodiments, it is envisioned that the formation of a more viscous composition will be advantageous in that it will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

V. Variants of Proteinaceous Compositions

Amino acid sequence variants of the proteins, polypeptides and peptides of the present invention can be substitutional, insertional or deletion variants, for example. Deletion variants lack one or more residues of the native protein that are not essential for function or immunogenic activity and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, may be employed in the invention.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the following exemplary changes: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of the peptide mimetics provided the biological activity of the mimetic is maintained. (see Table 1, below for a list of functionally equivalent codons).

TABLE 1

Codon Table

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include the following: arginine and lysine; glutamate and aspartate; serine and threonine; glutamine and asparagine; and valine, leucine and isoleucine.

VI. Pharmaceutical Compositions

In another aspect, the invention provides pharmaceutical compositions comprising a compound of the invention, or a pharmaceutically-acceptable salt thereof, and a pharmaceutically-acceptable carrier.

The pharmaceutical compositions of the invention comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

The phrase “therapeutically effective amount” as used herein means that amount of a compound, material, or composition comprising a compound of the present invention that is effective for producing some desired therapeutic effect, e.g., treating (i.e., preventing and/or ameliorating) cancer in a subject, or inhibiting protein-protein interactions mediated by an SH2 domain in a subject, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present compounds can contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound of the invention in the proper medium. Absorption enhancers can also be used to increase the flux of the compound of the invention across the skin. The rate of such flux can be controlled by either providing a rate controlling membrane or dispersing the compound of the invention in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral or topical administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the derivative (e.g., ester, salt or amide) thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, doses of the compounds of this invention for a patient, when used for the indicated effects, will range from about 0.0001 to about 100 mg per kilogram of body weight per day, more preferably from about 0.01 to about 50 mg per kg per day, and still more preferably from about 0.1 to about 40 mg per kg per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

VII. Mutagenesis

In certain aspects of the invention, a molecule is mutagenized, such as a molecule that upon mutagenesis becomes a Stat3 inhibitor, for example. Where employed, mutagenesis will be accomplished by a variety of standard mutagenic procedures. Mutation can involve modification of the nucleotide sequence of a single polynucleotide, such as a gene, blocks of polynucleotides, including genes, or whole chromosomes. Changes in single genes may be the consequence of point mutations that involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome, for example. They also are induced following exposure to chemical or physical mutagens. Such mutation-inducing agents include ionizing radiations, ultraviolet light, and a diverse array of chemical such as alkylating agents and polycyclic aromatic hydrocarbons, all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods, for example.

A. Random Mutagenesis

1. Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations (Oppenheimer et al. 1991). Insertion mutagenesis has been very successful in bacteria and Drosophila (Cooley et al. 1988) and recently has become a powerful tool in corn (Schmidt et al. 1987); Arabidopsis; (Marks et al., 1991; Koncz et al. 1990); and Antirrhinum (Sommer et al. 1990).

Transposable genetic elements are DNA sequences that can move (transpose) from one place to another in the genome of a cell. The first transposable elements to be recognized were the Activator/Dissociation elements of Zea mays (McClintock, 1957). Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini. these terminal inverted repeats may be anything from a few bases to a few hundred bases long and in many cases they are known to be necessary for transposition.

Prokaryotic transposable elements have been most studied in E. coli and Gram negative bacteria, but also are present in Gram positive bacteria. They are generally termed insertion sequences if they are less than about 2 kB long, or transposons if they are longer. Bacteriophages such as mu and D108, which replicate by transposition, make up a third type of transposable element. elements of each type encode at least one polypeptide a transposase, required for their own transposition. Transposons often further include genes coding for function unrelated to transposition, for example, antibiotic resistance genes.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another.

Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

2. Chemical Mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test (McCann et al., 1975) which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

3. Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman et al., 1989). Synthesis of cyclin and co-regulated polypeptides is suppressed by ionizing radiation in rat REF52 cells, but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte, et al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

4. In vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

In recent years, techniques for estimating the equilibrium constant for ligand binding using minuscule amounts of protein have been developed (Blackburn et al., 1991; U.S. Pat. Nos. 5,221,605 and 5,238,808). The ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

5. Random Mutagenesis by Fragmentation and Reassembly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

B. Site-Directed Mutagenesis

Structure-guided site-specific mutagenesis represents a powerful tool for the dissection and engineering of protein-ligand interactions (Wells, 1996, Braisted et al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166, for example.

VIII. Screening For Stat3 Inhibitors

The present invention further comprises methods for identifying modulators of the function of Stat3 and, in specific embodiments, for identifying an inhibitor of Stat3 activity. These assays may comprise random screening of large libraries of candidate substances; alternatively, the assays may be used to focus on particular classes of compounds selected with an eye towards structural attributes that are believed to make them more likely to modulate the function of Stat3.

By function, it is meant that one may assay for Stat3 interaction with other molecules, such as through the SH2 domain, for example, and/or for Stat3 phosphorylation, and/or Stat3 DNA binding activity, and/or the ability of Stat3 to translocate to the nucleus, and/or the ability of Stat3 to binding DNA and/or the ability of Stat3 to activate known Stat3 gene targets. In particular aspects of the invention, one may assay for the binding of Stat3 to a receptor, another Stat3 molecule, or both, for example. One or more candidate molecules may be identified or initially or further characterized by computer methods to assist in identifying appropriate configuration of the candidate molecule.

To identify a Stat3 modulator, one generally will determine the function of Stat3 in the presence and absence of the candidate substance, a modulator defined as any substance that alters function. For example, a method generally comprises:

(a) providing a candidate modulator;

(b) admixing the candidate modulator with an isolated compound or cell, or a suitable experimental animal;

(c) measuring one or more characteristics of the compound, cell or animal in step (c); and

(d) comparing the characteristic measured in step (c) with the characteristic of the compound, cell or animal in the absence of said candidate modulator,

wherein a difference between the measured characteristics indicates that said candidate modulator is, indeed, a modulator of the compound, cell or animal.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

A. Modulators

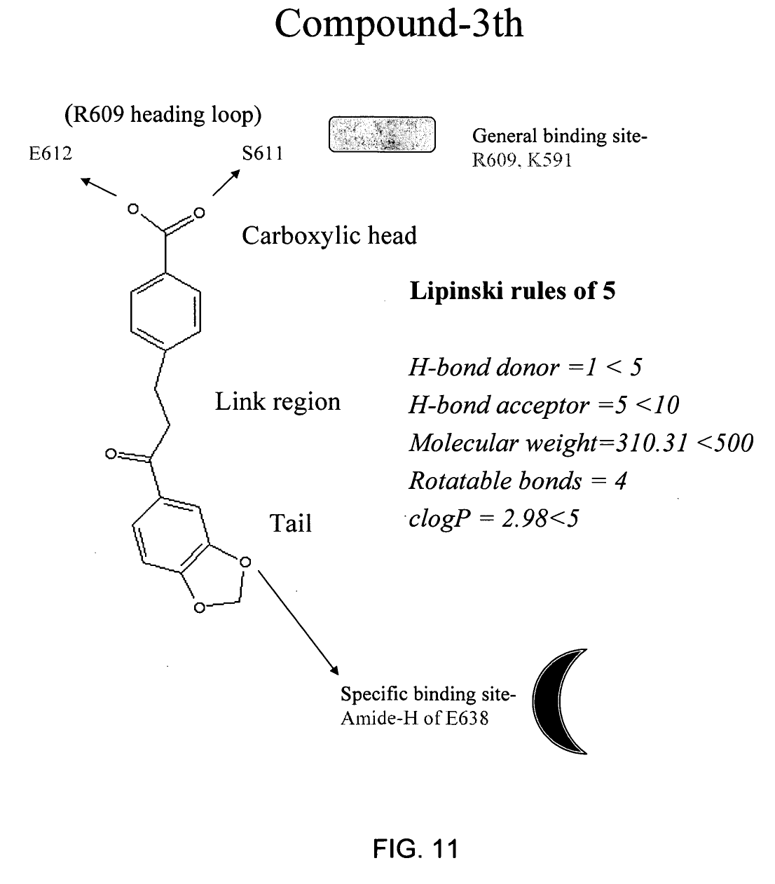

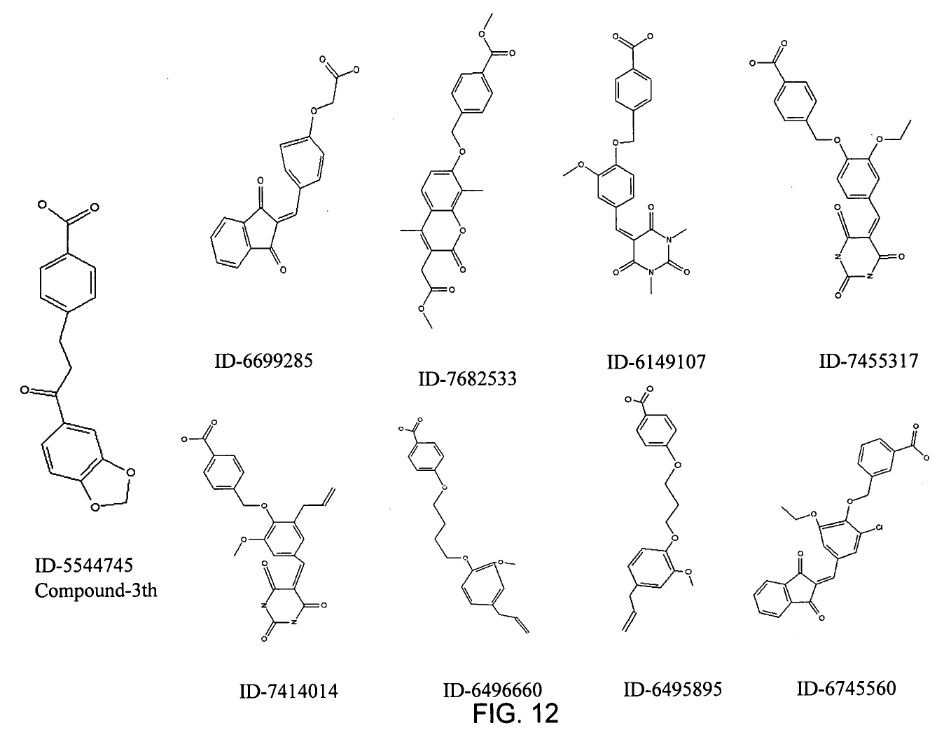

As used herein the term “candidate substance” refers to any molecule that may potentially inhibit or enhance Stat3 activity. The candidate substance may be a protein or fragment thereof, a small molecule, or even a nucleic acid molecule, for example. It may prove to be the case that the most useful pharmacological compounds will be compounds that are structurally related to beta turn mimetics capable of binding at least part of the SH2 domain or that are structurally related to the compound of FIG. 11. Using lead compounds to help develop improved compounds is known as “rational drug design” and includes not only comparisons with known inhibitors and activators, but predictions relating to the structure of target molecules.

The goal of rational drug design is to produce structural analogs of biologically active polypeptides or target compounds. By creating such analogs, it is possible to fashion drugs that are more active or stable than the natural molecules and that have different susceptibility to alteration or that may affect the function of various other molecules. In one approach, one would generate a three-dimensional structure for a target molecule, or a fragment thereof. This could be accomplished by nuclear magnetic resonance, x-ray crystallography, computer modeling or by a combination of these approaches, for example.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on Stat3. Regardless of the type of inhibitor or activator identified by the present screening methods, the effect of the inhibition or activator by such a compound results in inhibition of Stat3 activity as compared to that observed in the absence of the added candidate substance.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

C. In Cyto Assays

The present invention also contemplates the screening of compounds for their ability to modulate Stat3 in cells. Various cell lines can be utilized for such screening assays, including cells specifically engineered for this purpose.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that inhibits Stat3. In these embodiments, the present invention is directed to a method for determining the ability of a candidate substance to inhibit Stat3, generally including the steps of: administering a candidate substance to the animal; and determining the ability of the candidate substance to reduce one or more characteristics of Stat3.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Site-Directed Mutagenesis of Stat3

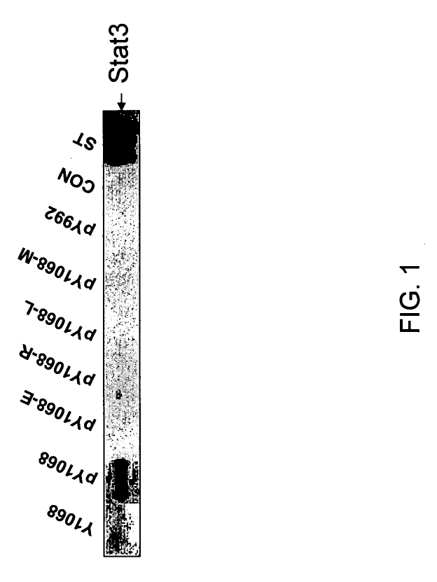

Human Stat3α cDNA was a gift from Dr. Rolf Van de Groot (also see Rahuel et. al, 1998). A HindIII/XhoI DNA fragment containing Stat3α was cloned into the baculovirus expression vector, pFastBac1 (Invitrogen, GIBCO; Carlsbad, Calif.) with a 6-histidine tag engineered onto the N terminus of human Stat3. Single or combination mutations were generated using Quikchange site-directed mutagenesis kit (Stratagene; La Jolla, Calif.) to target amino acid residues within the Stat3 SH2 domain implicated in models of Stat3 SH2-phosphotyrosine binding (K591L, R609L, E638P, E638L, Y640F, Y657F, C687A, S691A and Q692L; FIG. 1). The sequence of each construct was verified by sequencing analysis.

### Example 2

### Expression and Purification of Stat3 Proteins

The wild type and mutated Stat3 plasmid was used to transform DH10Bac competent cells, which contain a bacmid with a mini-attTn7 target site and helper plasmid. Recombinant bacmids were prepared and used to infect Sf9 cells. Sf9 cells (3×10⁶ cells per ml) were infected with Stat3 recombinant virus at a multiplicity of infection of 0.05 and harvested after 3-day culture. Cells (6×10⁸) were suspended in 12 ml pre-cooled lysis buffer (20 mM Tris-Cl pH8.0, 0.5M NaCl, 10% glycerol, 1 mM phenylmethylsulfonyl fluoride, 10 ug/ml leupeptin, 1 ug/ml aprotinin, 10 mM imidazole) and lysed by ultrasonication on ice. Lysates were centrifuged at 15,000 g for 30 min at 4° C. and the supernatant was incubated with Ni-NTA agarose (QIAGEN) at 4° C. for 1 hr. The Ni-NTA resin was washed twice with 4 volumes of wash buffer (20 mM Tris-Cl pH8.0, 0.5M NaCl, 10% glycerol, 1 mM phenylmethylsulfonyl fluoride, 10 ug/ml leupeptin, 1 ug/ml aprotinin, 20 mM imidazole) to remove unbound proteins. Stat3 was eluted from the Ni-NTA resin with elution buffer (20 mM Tris-Cl pH8.0, 0.5M NaCl, 110% glycerol, 1 mM phenylmethylsulfonyl fluoride, 10 ug/ml leupeptin, 1 ug/ml aprotinin, 250 mM imidazole). The purified proteins were dialyzed against 10 mM PBS at 4° C. and stored at −80° C.

### Example 3

### Peptide Synthesis

The exemplary peptides listed in Table 2 were synthesized in the Baylor College of Medicine Protein Core Facility on an Applied Biosystems (Foster City, Calif.) Model 433A peptide synthesizer using standard 9-fluorenylmethoxycarbonyl amino acid chemistry. Seventy percent of the peptide reaction mix was biotinylated at the N-terminus while the peptide remained on the resin using d-Biotin-LC (AnaSpec, Inc.). All peptides were purified using reverse-phase high performance liquid chromatography and were ≧95% pure.

### Example 4

### Phosphopeptide Affinity Immunoblot Analysis

NeutrAvidin agarose (40 μl; Pierce) was incubated with 10 μg of biotinylated peptide in 300 μl of Buffer A (20 mM HEPES pH 7.5, 20 mM NaF, 1 mM Na₃VO₄, 1 mM Na₄P₂O₇, 1 mM EDTA, 1 mM EGTA, 20% glycerol, 0.05% NP-40, 1 mM DTT, 1 μg/ml leupeptin, 1 μg/ml aprotinin, 0.5 mM phenylmethylsulfonyl fluoride, 100 mM NaCl) at 4° C. for 2 h and washed with Buffer A 3 times. The NeutrAvidin-peptide complex was then mixed with His-tagged Stat3 protein (5 μg) in 1 ml of Buffer A (without NaCl and NP-40) at 4° C. for 2 h and washed thoroughly. Bound proteins were separated and immunoblotted using Stat3 monoclonal antibody (mAb).

### Example 5

### Mirror Resonance Affinity Assay

Kinetics experiments were performed using an lasys Auto+ resonant mirror biosensor (Affinity Sensor, Paramus, N.J.) (see Sadowski et. al, 1986). Briefly, two-welled cuvettes coated on the bottom of each well with biotin were purchased from Affinity Sensor and prepared for immobilization of biotinylated peptides by coating each surface with 0.04 mg/ml NeutrAvidin (Pierce) and washing with PBS-T (20 mM Na Phosphate, 0.05% Tween-20). Biotinylated peptide (5 μg) was added into each well—experimental peptide to one well and control peptide to the other—and change in arc seconds monitored simultaneously in both wells using the biosensor until stable followed by washing with PBS-T. Real-time binding of Stat3 was conducted at 25° C. at a stir speed of 70 for 10 min starting at the lowest concentration of Stat3. The wells were washed out with three changes of 60 μl PBS-T, and dissociation was allowed to proceed for 5 min. Each well bottom was regenerated by washing with 5011 of 100 mM formic acid for 2 min and equilibrated with PBS-T for the next round of association assay. Data were collected automatically and analyzed with the FASTplot and GraFit software (see Sheinerman et. al, 2003).

### Example 6

### Co-EXPRESSION of G-CSFR and Stat3 in 293T Cells

Hind III/XhoI cDNA fragments encoding His-tagged wild type and mutant Stat3 were subcloned into pcDNA3.1(−) (Invitrogen). The full-length human G-CSF receptor cDNA was a gift from Dr. Steven F. Ziegler (Ziegler et al., 1991). Both the G-CSF receptor and Stat3 vectors were co-transfected into 293T cells using Fugene6 (Roche) and incubated for 48 h. The cells were starved for 6 h and then stimulated with 100 ng/ml of G-CSF (R & D Systems; Minneapolis, Minn.) for 15 min.

For immunoprecipitation, cells were placed in lysis buffer (50 mM Tris-HCl, 150 mM NaCl, 1% NP-40, 1 mM EDTA, 0.25% sodium deoxycholate, 1 mM phenylmethylsulfonyl fluoride, 10 ug/ml leupeptin and 10 ug/ml aprotinin) and sonicated. Lysate supernatants were incubated with anti-G-CSF receptor antibody (CD114, RDI, Inc.) at 4° C. for 1 h followed by incubation with protein-G Sephorose (Sigma) for 2 h. Immunoprecipitates were washed five times with lysis buffer then boiled for 5 min in SDS-PAGE sample buffer. For Ni-His tagged protein pull-down assay, cells were placed in cell suspension buffer (20 mM Tris-Cl pH8.0, 0.5M NaCl, 10% glycerol, 1 mM phenylmethylsulfonyl fluoride, 10 ug/ml leupeptin, 1 ug/ml aprotinin, 10 mM imidazole) and lysed by ultrasonication on ice. The supernatant was incubated with Ni-NTA agarose (Qiagen) at 4° C. for 2 hr. The Ni-NTA agarose was washed five times with cell suspension buffer containing 20 mM imidazole to remove unbound proteins then boiled for 5 min in SDS-PAGE sample buffer. Immunoprecipitates and Ni-NTA pull-downs were separated on SDS-PAGE gels and transferred to polyvinylidene difluoride membranes. G-CSF receptor was detected by anti-human G-CSFR antibody (R&D systems). Total Stat3 was detected as described above; Y705 phosphorylated State was detected using antibodies purchased from BD Transduction Laboratories or Cell Signaling Technology.

### Example 7

### Circular Dichroism (CD)

CD spectra of the WT and E638P mutants of Stat3 were recorded between the 280 to 190 nm range in 10 mM phosphate-buffered saline on an Olis DSM 1000 CD spectrophotometer. Measurements were performed at a protein concentration of 1.8 μM and 1.6 μM for the WT and mutant Stat3, respectively, using a 1 mm cuvette. Spectra were acquired at 10° C. with a 2 s integration time and repeated three times for each sample.

### Example 8

### Requirement for +3 Q within the Y1068 Phosphopeptide Ligand for Stat3 Binding

Peptide affinity immunoblot analysis and mirror resonance imaging studies using phosphorylated and non-phosphorylated dodecapeptides based on the amino acid sequence within the region of the EGFR containing Y1068 and Y1086 demonstrated the requirement for their phosphorylation on tyrosine to achieve measurable binding of native and recombinant Stat3. These studies also revealed that Y1068 phospododecapeptide bound with 2-fold higher affinity than Y1086 PDP.

To determine whether or not the polar residue Q at the +3 position of pY1068 peptide is essential for Stat3 SH2 binding, a panel of tyrosine phosphorylated dodecapeptides based on Y1068 were synthesized in which +3 Q was left unchanged or replaced by a residue with a non-polar side chain L or M, an acidic side chain E, or a basic side chain R (Table 2).

TABLE 2

IX. Tyrosine phosphorylated and
non-phosphorylated peptides
synthesized based upon the EGFR
sequence

| | | |
|---|---|---|
| PEPTIDE | SEQUENCE | SEQ ID NO: |

| | | |
|---|---|---|
| pY992 | TDSNF(pY)RALMDE | 5 |
| pY1068 | LPVPE(pY)INQSVP | 3 |
| pY1068-R | LPVPE(pY)INRSVP | 6 |
| pY1068-E | LPVPE(pY)INESVP | 7 |
| pY1068-M | LPVPE(pY)INMSVP | 8 |
| pY1068-L | LPVPE(pY)INLSVP | 9 |
| Y1068 | LPVPEYINQSVP | 10 |
| pY1086 | VQNPV(pY)HNQPLN | 4 |
| Y1086 | VQNPVYHNQPLN | 11 |
| pY1148 | VGNPE(pY)LNTVQP | 12 |
| pY1173 | LDNPD(pY)QQDFFP | 13 |

Each peptide was incubated with equal amounts of purified wild type Stat3 protein in peptide pull-down assays (FIG. 1). NeutrAvidin agarose was incubated with the indicated biotinylated peptides (see Table 2 for sequence) or no peptide (CON) as control, washed thoroughly and mixed with identical amount of wild type Stat3. Bound proteins were separated by SDS-PAGE and immunoblotted using Stat3 mAb. Lane ST represents purified wild type Stat3 (0.6 μg) loaded directly onto the gel as positive control. Immunoblotting for Stat3 demonstrated a prominent Stat3 band in pull-down assays using wild type Y1068 PDP. In contrast, little to no Stat3 was detected in pull-down assays using PDPs in which the Q was mutated to L, M, E or R similar to results using unphosphorylated Y1068 dodecapeptide. Thus, Q at the +3 position of Y1068 phosphopeptide is required for Stat3 binding and appears to be as important for Stat3 binding as phosphorylation on tyrosine. Real-time resonance mirror affinity assays using Y1068 Q-to-R PDP, which was the only PDP to demonstrate any detectable binding of Stat3 in peptide immunoblot studies, demonstrated that mutation of Q to R decreased Stat3 binding to undetectable.

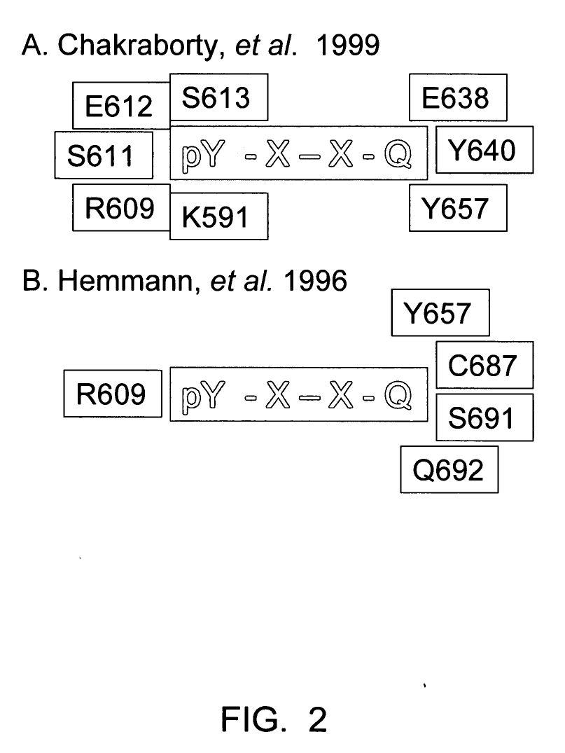

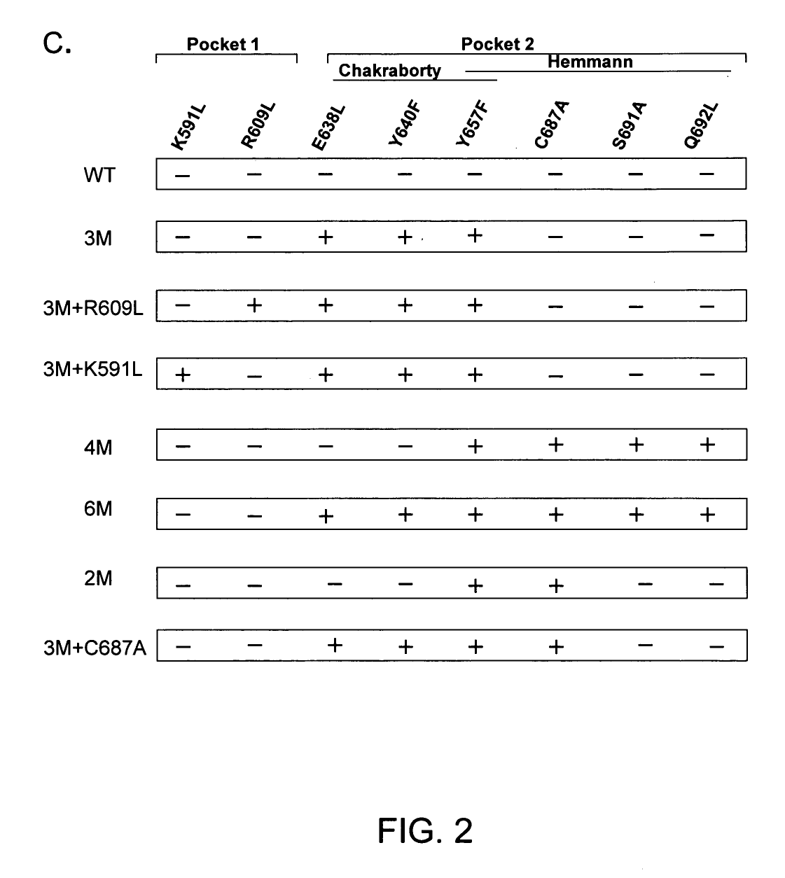

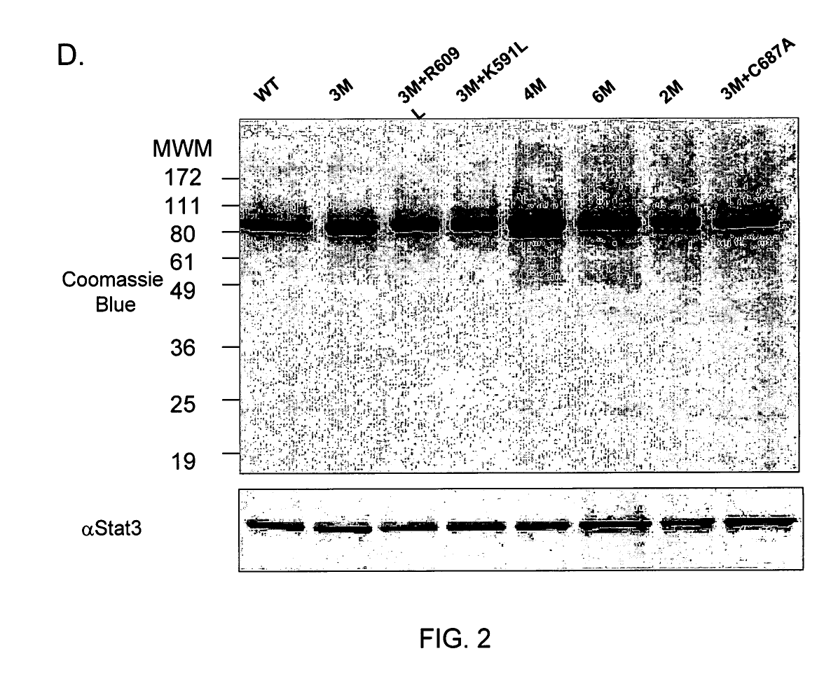

The side chains of K591 and R609 within pocket 1 of Stat3, but not the side chains of amino acid residues within pocket 2, are essential for Stat3 binding to YXXQ-containing phosphopeptides. Hemmann et al. and Chakraborty et al. previously proposed two distinct but overlapping two-pocket models for the binding of YXXQ-containing PDP ligands by the Stat3 SH2 domain; both models assumed the peptide ligand was in an extended configuration (FIGS. 2A and 2B). The phosphotyrosine residue interacts with a positively charged pocket (pocket 1) within the SH2 domain formed primarily by the side chains of K591 and R609 and secondarily by the side chains of S611, E612 and S613. The pY +3 Q was predicted to interact with a hydrophilic pocket (pocket 2) formed by the side chains of E638, Y640 and Y657. In the Hemmann model, the phosphotyrosine was predicted to interact with the side chain of R609 (pocket 1) and the +3 Q with the side chains of Y657, C687, S691 and Q692 (pocket 2).

In order to test each of the two models proposed, Stat3 mutants were generated in which mutations were introduced to change charged or polar side chains to non-polar within amino acid residues predicted in each model to be critical for Stat3 binding (FIG. 2C). His tags were added at the N terminus of each protein to aid in purification; the recombinant Stat3 proteins were expressed in Sf9 insect cells and purified to equivalent levels using Ni-NTA resin (FIG. 2D). Wild type and mutant Stat3 proteins, each with an N-terminal His-tag, were expressed in SF9 insect cells and affinity purified using Ni-NTA agarose. The eluates were separated by SDS-PAGE and the gel stained with Coomassie Blue (top panel) or immunoblotted using Stat3 mAb (bottom panel)

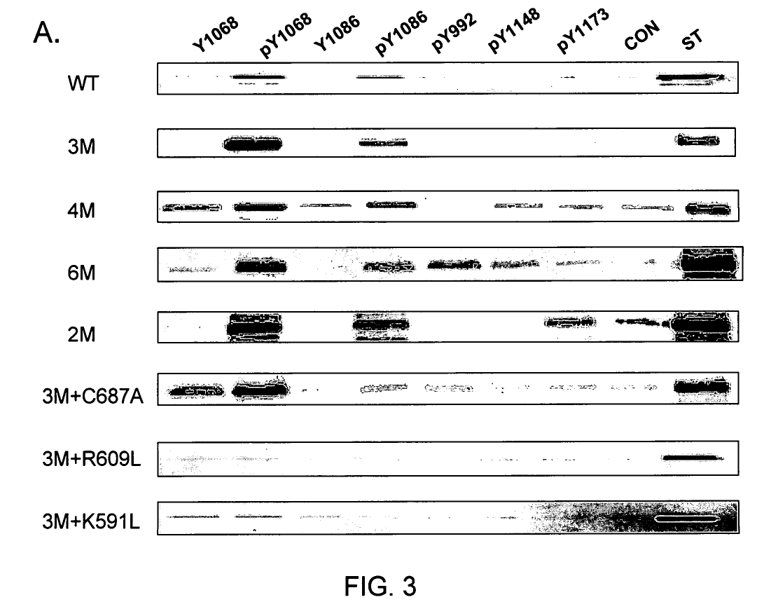

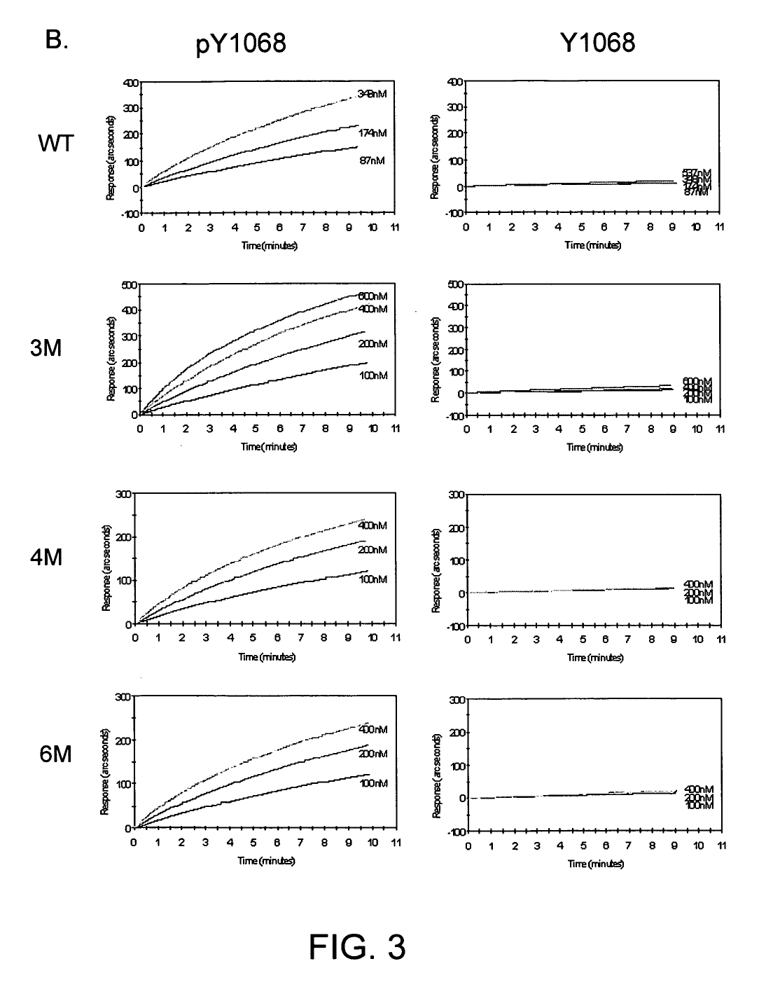

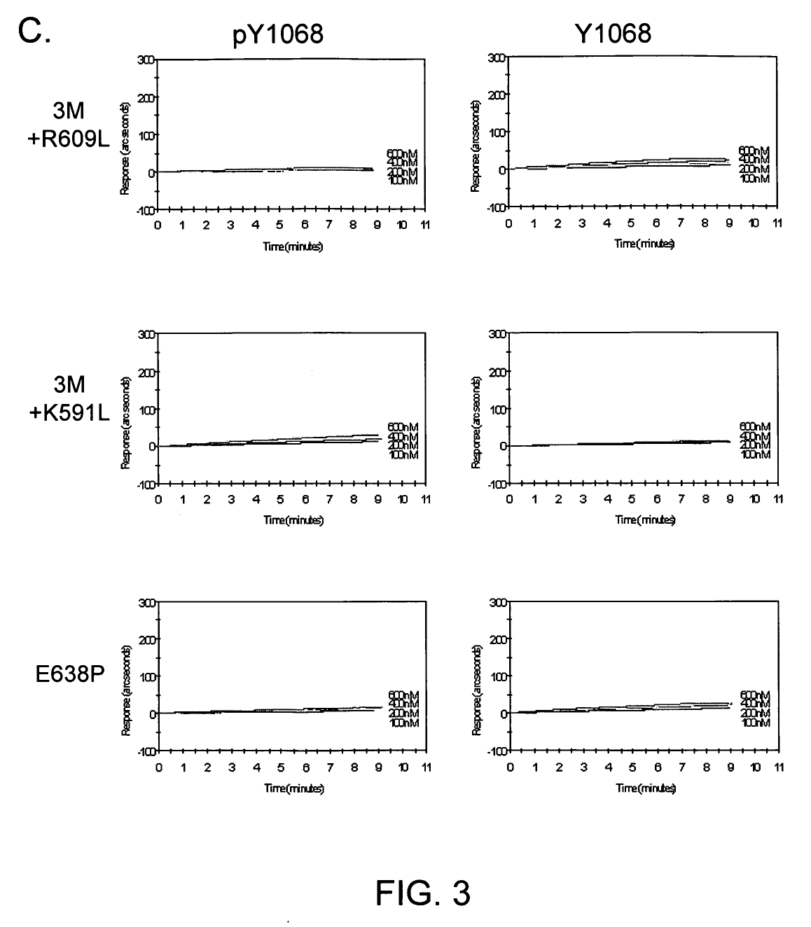

Peptide affinity immunoblot studies using Stat3-3M to test the pocket 2 component of the Chakraborty model demonstrated levels of Stat3-3M bound to Y1068 and Y1086 PDPs similar to wild type Stat3 (FIG. 3A). Peptide affinity immunoblot studies using Stat3-4M to test the pocket 2 component of the Hemmann model demonstrated levels of binding of Stat3-4M bound to Y1068 and Y1086 phosphopeptides equal to or greater than wild type Stat3 (FIG. 3A). Stat3-6M, in which all six amino acid residues predicted by both models to form pocket 2 were mutated, also bound both PDPs at levels similar to wild type Stat3 as did Stat3-2M and Stat3-3M+C687A. These results do not support either model for Stat3 SH2 binding to +3 Q within phosphopeptide ligands. NeutrAvidin agarose was incubated with the indicated biotinylated peptides (see Table 2 for exemplary sequences) or no peptide (CON) as control, washed thoroughly and mixed with identical amounts of wild type or mutant Stat3 proteins as indicated. Bound proteins were separated by SDS-PAGE and immunoblotted using Stat3 mAb. Lane ST represents purified wild type Stat3 (0.6 μg) loaded directly onto the gel as positive control.

To test the pocket 1 component of the two models and to ensure that the peptide pull-down system was sufficiently sensitive to detect reduced binding of Stat3 containing mutations in pocket 2, either K591L or R609L was added to the 3M mutant to generate Stat3-3M+K591L and Stat3-3M+R609L. Addition of either mutation resulted in elimination of binding to both Y1068 and Y1086 PDPs, indicating that each of the side chains of K591 and R609 make important contributions to binding of the phosphotyrosine.

To confirm these findings and to determine if introduction of the pocket 2 mutations resulted in subtle alterations in kinetics of binding undetectable using phosphopeptide affinity immunoblot analysis, mirror resonance affinity assays were performed using phosphorylated and non-phosphorylated Y1068 dodecapeptide (FIGS. 3B and 3C and Table 3). Mirror resonance affinity assay. Two cells of a biotin-coated cuvette pretreated with saturating amounts of NeutrAvidin. One well of the cuvette was pretreated with biotinylated phosphopeptide based on Y1068 (pY1068, left panel), while the other well was pretreated with biotinylated non-phosphorylated peptide Y1068 (Y1068, right panel) as a control for non-specific binding. Wild type or mutated Stat3 protein was added in the concentrations indicated to each of the two cells and mirror resonance measurements recorded continuously for 10 min as shown.

TABLE 3
X. Kinetics of wild type and mutant Stat3 binding to Y1068
PDP determined by mirror resonance biosensor analysis.

| Stat3 | kass(M − 1 s − 1)^{a} | kdiss(ms − 1)^{b} | KD(nM)^{c} |
|---|---|---|---|
| WT | 3073 | 0.7 | 223 |
| 3M | 3371 | 0.6 | 177 |
| 4M | 2673 ± 481_{d} | 0.8 ± 0.3 | 271 ± 48 |
| 6M | 2619 ± 674_{d} | 0.7 ± 0.3 | 249 ± 22 |

^{a}Association rate constant determined from slope of line from plot of kass vs. [ligand].
^{b}Dissociation rate constant determined from y intercept of plot of kass vs. [ligand].
^{c}Dissociation equilibrium constant determined from ratio of kdiss/kass.
^{d}Mean ± SEM of 2 separate experiments

Review of the real-time mirror resonance affinity curves (FIGS. 3B and 3C) and kinetic analysis (Table 3) revealed undetectable binding of Stat3-3M+K591 and Stat3-3M+R609 to phosphorylated Y1068 dodecapeptide confirming the results of peptide immunoblot analysis. Furthermore, each of the pocket 2 mutant Stat3 proteins examined (3M, 4M and 6M) demonstrated kₐₛₛ, k_{diss} and K_{D} values for binding to Y1068 PDPs indistinguishable from wild type Stat3 confirming the peptide immunoblot analysis and indicating that Stat3 SH2 binding to the +3 Q within Y1068 does not require any of the side chains predicted in either of the proposed models.

### Example 9

### Computational Modeling of Stat3 SH2 Binding to +3 Q within YXXQ-Containing Phosphopeptides

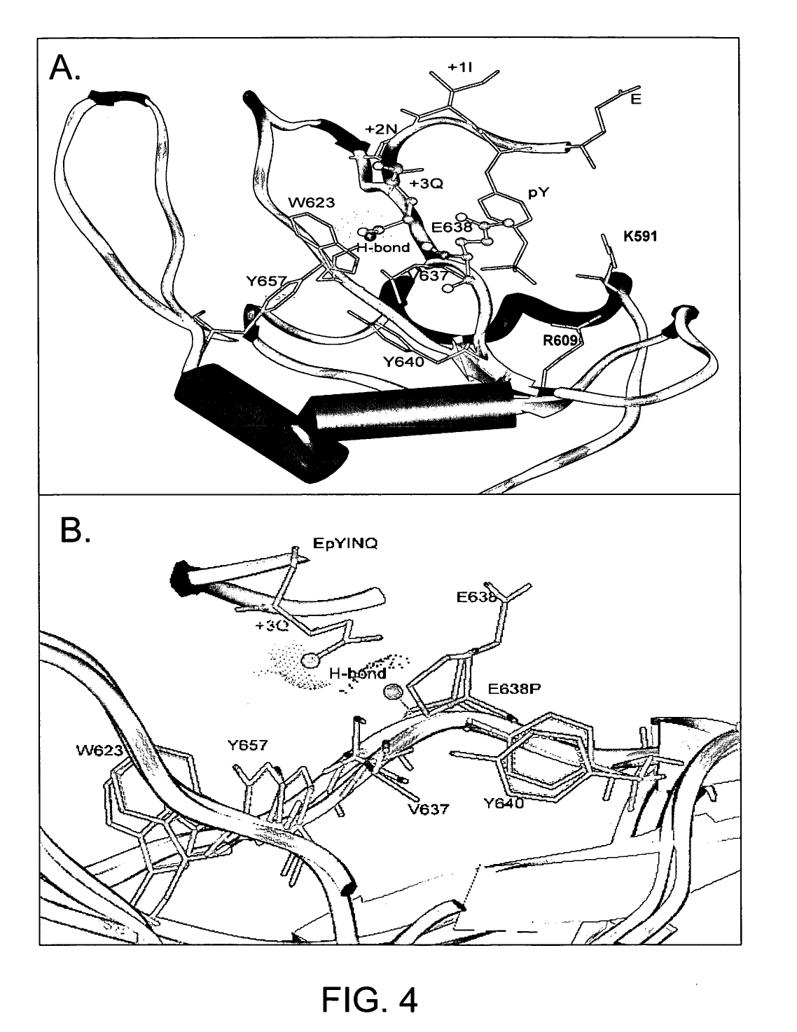

To generate a new and more accurate model for Stat3 SH2 binding to +3 Q, the structure of Y1068 phosphopeptide was used (EpYINQ), which was available from its crystal structure bound by Grb2 (Kuriyan et. al, 1997) (PDB code 1ZFP) and the structure of Stat3 from W580 to L670; it was obtained from the crystal structure of Stat3β bound to DNA (Becker et. al, 1998) (PDB code 1BG1) to computationally model the interaction with the lowest energy. All energy minimization calculations were carried out under AMBER force field by using the DISCOVER/Insight II program. A total of 300 steps of conjugate gradient energy minimization were performed following rigid hand-docking to fit the pY of the EpYINQ peptide into the binding pocket comprised of residues K589 and R607 taking into consideration Van der Waals and Coulomb forces. The interaction between Stat3-SH2 and EpYINQ with the complex lowest energy (FIG. 4A) had a total binding energy of −478.8 Kcal/mol. As indicated, the oxygen on the side chain of the pY +3 Q within the EpYINQ peptide is predicted to form a hydrogen (H) bond with the amide hydrogen at E638 and to make a major contribution to the binding energy. The positions are shown for the side chains of K589 and R607 proposed to be major contributors to pocket 1, E638, Y640 and Y657 proposed by Chakraborty to form pocket 2 and for the side chain of W623 proposed to force a P turn in the peptide ligand. The +3 Q and E638 are shown as ball-and-stick models, the remaining side chains as stick models; oxygen atoms are shown in red, carbon in gray, nitrogen in blue and phosphorus in orange. This computational result predicted that the major binding energy for this binding configuration comes from a hydrogen bond interaction involving oxygen within the pY +3 Q side chain and the peptide amide hydrogen at E638 located within a loop region of Stat3 SH2.

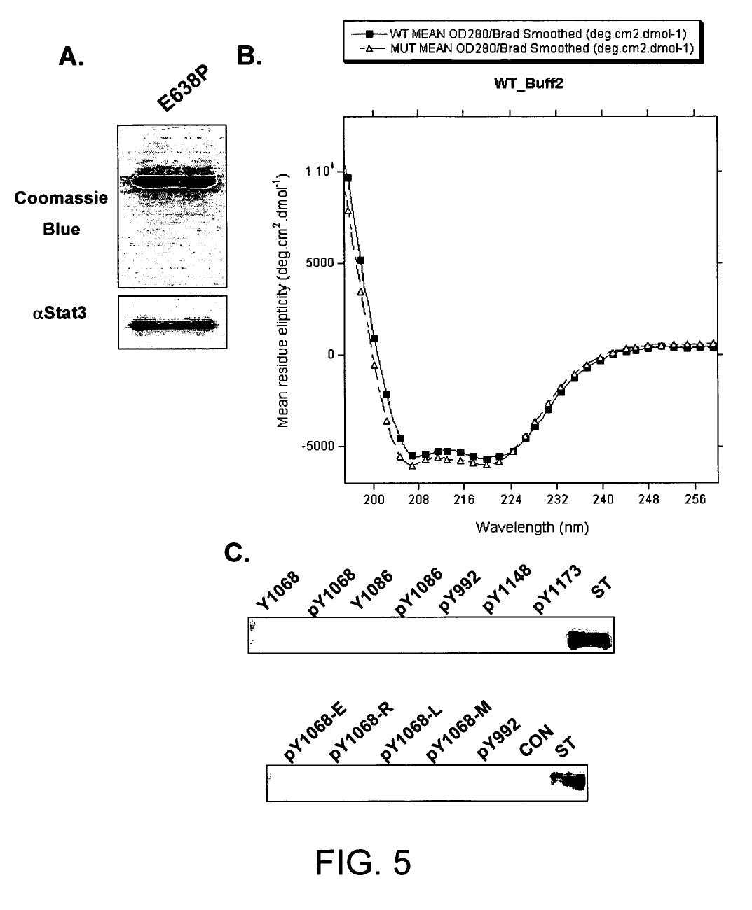

To test the contribution of the E638 amide hydrogen, Stat3-E638P was generated by site-directed mutagenesis, which eliminated the amide hydrogen donor predicted to bind with oxygen within the +3 Q side chain. In consideration of the possible effect of this mutation on secondary structure, E638P was modeled within Stat3-SH2 using Biopolymer in the Insight II environment and carried out local energy minimization: 1) with all residues fixed except for V637 to P639 to assess the effect in the immediate vicinity of the E638P mutation and 2) will all residues fixed except for residues from 1628 to M648 to assess the effect of E638P on structure further N- and C-terminal to E639P. When the resultant structures were overlaid onto the wild type Stat3 there was no physical differences between the two structures with the exception of a slight reduction in the angle of the backbone loop turn at residues V637, E638P and P639 (FIG. 4B). Recombinant Stat3-E638P was produced in Sf9 cells. It was expressed to levels similar to wild-type Stat3 (FIG. 5A) and demonstrated solubility characteristics similar to wild type Stat3. Furthermore, circular dichroism (CD) analysis of Stat3-E638P (FIG. 5B) revealed a folded protein with a predominantly alpha-helical structure essentially identical to wild type Stat3 confirming and strengthening the conclusions reached from computational modeling that introduction of the E639P mutation does not result in unanticipated local or global secondary structural changes.

Peptide affinity immunoblot assays demonstrated no binding of Stat3-E638P to any of the EGFR derived peptides tested including Y1068 and Y1086 PDP (FIG. 5C); mirror resonance affinity studies (FIG. 3C) confirmed these findings. These results strongly supported an important role for the E638 amide hydrogen of Stat3 in binding of the +3 Q within Y1068 PDP. In FIG. 5C, NeutrAvidin agarose was incubated with the indicated biotinylated dodecapeptides (see Table 2 for sequence) or no peptide (CON) as control, washed thoroughly and mixed with wild type Stat3 protein. Bound proteins were separated by SDS-PAGE and immunoblotted using Stat3 mAb. Lane ST represents purified wild type Stat3 (0.6 μg) loaded directly onto the gel as a positive control.

### Example 10

### Stat3 Binds Directly to G-CSFR Y704 and Y744 Phosphododecapeptides

Studies using the M1 cell line containing wild type G-CSFR constructs and constructs containing Y-to-F mutants at single and multiple Y residues within its C-terminal cytoplasmic domain indicated that G-CSF-mediated Stat3 activation mapped to Y704 and Y744. In addition, Stat3 destabilization and peptide affinity studies using phosphododecapeptides based upon each of the four pY sites within the G-CSFR indicated that only Y704 and Y744 were able to destabilize Stat3 dimers and to affinity purify Stat3 from whole cell extracts. Ward et al. confirmed the Stat3 destabilization results using phosphopeptides that were nine residues in length and based on the four pY sites within the murine G-CSFR; they also demonstrated direct binding of a GST-Stat3 SH2 domain fusion protein to the phosphorylated cytoplasmic domain of the human G-CSFR, which indicated that that the interaction was mediated through the Stat3 SH2 domain. Recombinant human Stat3 protein was generated with His tags added at the N terminus to aid in purification; this modification did not interfere with binding of wild type Stat3 to native full-length, activated EGFR or to EGFR-derived phosphododecapeptides. Recombinant wild type Stat3 protein was expressed in Sf9 insect cells and purified using Ni-NTA resin.

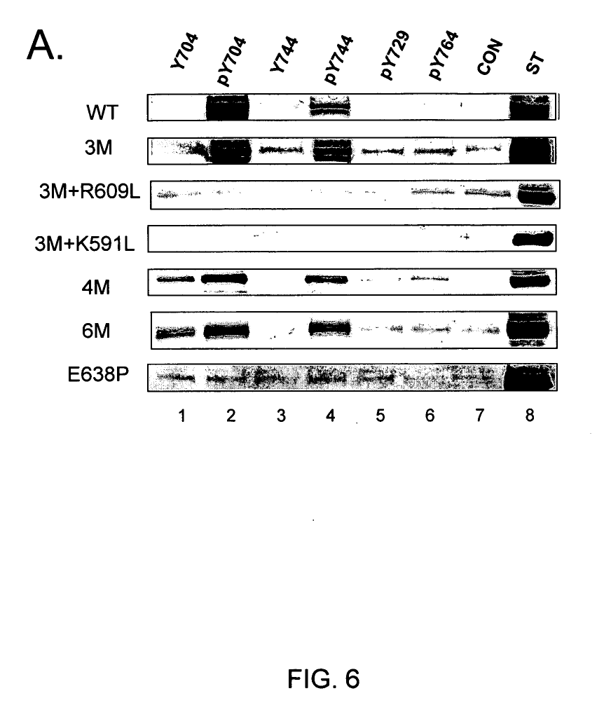

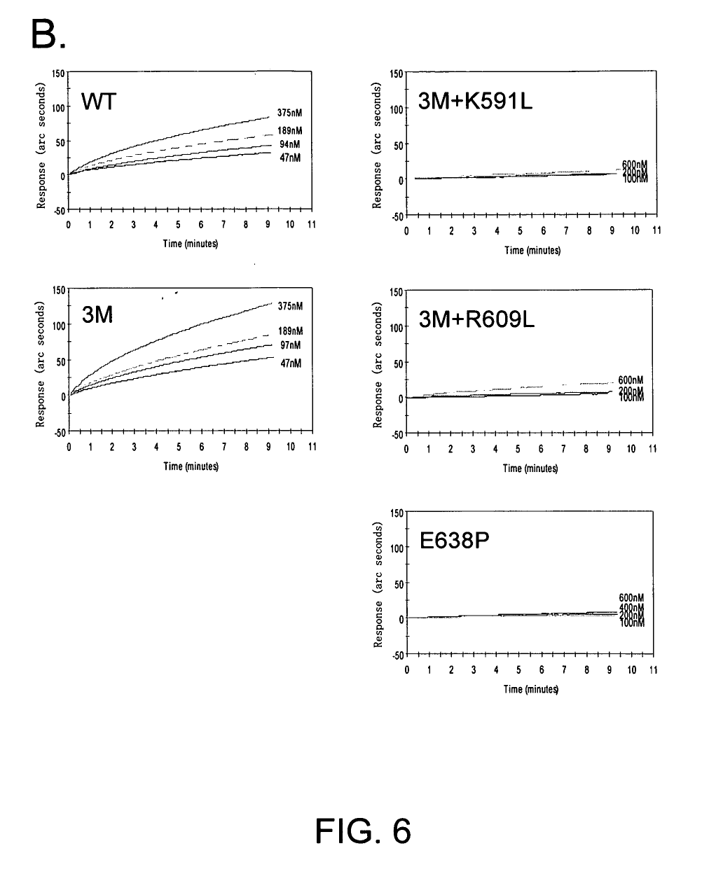

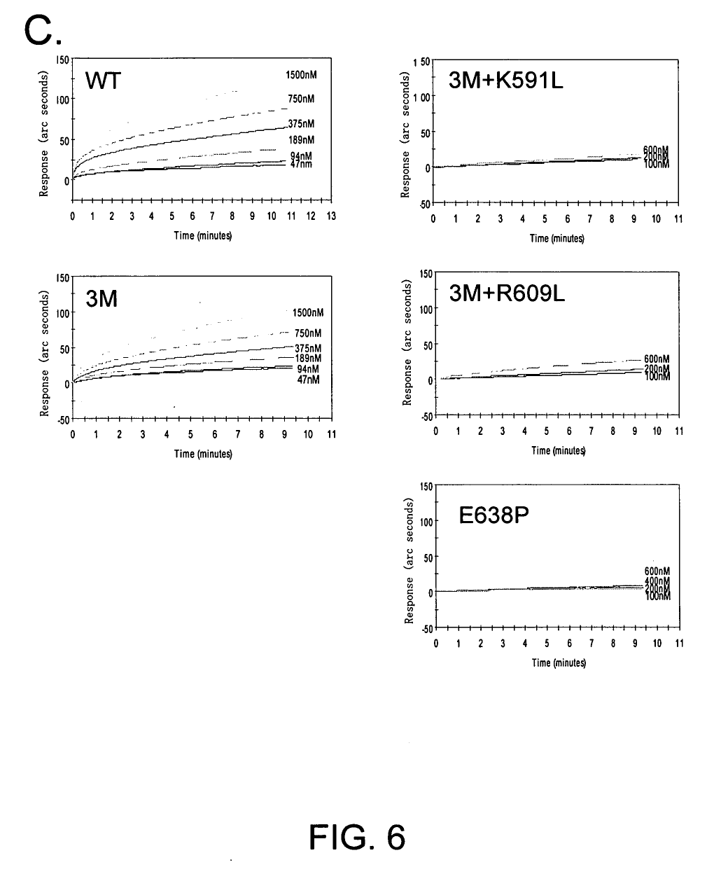

Purified Stat3 was incubated with phosphododecapeptides based on each of the four G-CSFR Y residues (Table 4) in pull-down assays (FIG. 6A). Immunoblotting for Stat3 demonstrated a prominent Stat3 band in pull-down assays using Y704 and Y744 phosphododecapeptide. Neither of the other two G-CSFR phosphododecapeptides bound purified Stat3 above control level. The ability of both Y704 and Y744 dodecapeptides to bind purified Stat3 depended on the tyrosine being phosphorylated.

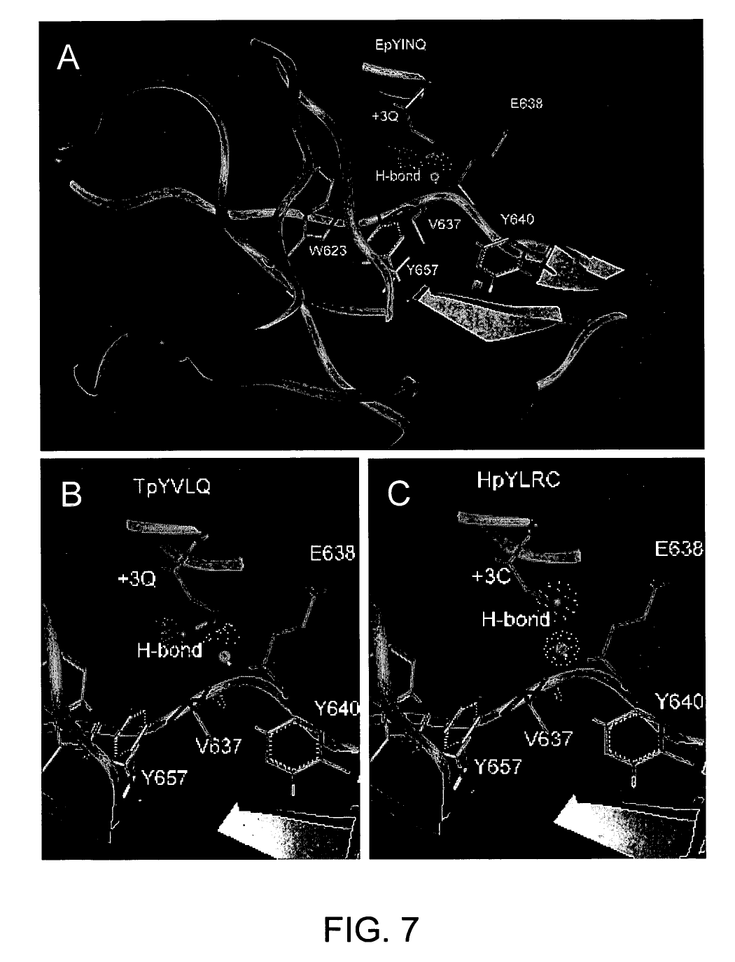

To obtain quantitative kinetic information about the binding of Stat3 to G-CSFR Y704 and Y744 including association rates (kₐₛₛ), disassociation rates (k_{diss}) and dissociation equilibrium constants (K_{D}), real-time affinity measurements using a mirror resonance biosensor were performed. The biosensor exploits surface plasmon resonance to measure in real time the alteration in the angle of a laser light reflected from a surface upon which binding events are occurring. Biotinylated peptides were immobilized onto the bottom surface of cuvette wells pre-coated with NeutrAvidin. The interaction of peptides with Stat3 added at different concentrations was measured in real time as altered deflection of a laser light striking the bottom surface of the cuvette; the alterations in the deflection angle measured in arc seconds was analyzed with GraFit software. Mirror resonance analysis (FIGS. 7B and 7C and Table 5) demonstrated that Stat3 bound to phosphododecapeptide Y704 with a K_{D} of 0.703 μM, similar to phosphododecapeptide Y744, which demonstrated a K_{D} of 0.95 μM. The slightly lower K_{D} for Y704 vs. Y744 is attributable to a faster association rate of Stat3 binding to this phosphododecapeptide.

TABLE 4

XI. Exemplary tyrosine
phosphorylated and non-
phosphorylated peptides
synthesized based upon the G-CSFR
sequence.

| | |
|---|---|
| Peptide | Amino Acid Sequence |

| | | | |
|---|---|---|---|
| XII. | Y704 | TLVQTYVLQGDP | SEQ ID NO:18 |
| XIII. | pY704 | TLVQTpYVLQGDP | SEQ ID NO:17 |
| XIV. | pY729 | SDQVLpYGQLLGS | SEQ ID NO:21 |
| XV. | Y744 | PGPGHYLRCDST | SEQ ID NO:20 |
| XVI. | pY744 | PGPGHpYLRCDST | SEQ ID NO:19 |
| XVII. | pY764 | PSPLSpYENLTFQ | SEQ ID NO:22 |

TABLE 5
XVIII. Kinetics of wild type and mutant Stat3 binding
to Y704 and Y744 phosphododecapeptides (PDP) determined
by mirror resonance biosensor analysis.

| XIX. PDP | Stat3 | kₐₛₛ(M⁻¹s⁻¹) ^{a} | k_{diss}(ms⁻¹)^{b} | K_{D}(μM) ^{c} |
|---|---|---|---|---|
| XX. 704 | WT | 2298 | 1.6 | 0.703 |
| XXI. | 3M | 1503 ± 208 ^{d} | 1.9 ± 0.3 | 1.21 ± 0.01 |
| XXII. 744 | WT | 1413 ± 324 ^{d} | 1.4 ± 0.4 | 0.95 ± 0.14 |
| | 3M | 1470 ± 716 ^{d} | 2.1 ± 0.7 | 1.06 ± 0.15 |

^{a} Association rate constant determined from slope of line from plot of kₐₛₛ vs. [ligand].
^{b} Dissociation rate constant determined from y intercept of plot of kₐₛₛ vs. [ligand].
^{c} Dissociation equilibrium constant determined from ratio of k_{diss}/kₐₛₛ.
^{d} Mean ± SEM of 2 or more separate experiments.

### Example 11

### The Side Chains of K591 and R609 within Pocket 1 of Stat3, But not the Side Chains of Amino Acid Residues within Pocket 2, are Essential for Stat3 Binding to Y704 and Y744 Phosphododecapeptides

A two-pocket model for the binding of G-CSFR Y704 and Y744 phosphopeptide ligands by the Stat3 SH2 domain (FIG. 1A) that was distinct yet had overlapping features with that proposed by Hemmann et al. for binding of Stat3 SH2 to pY ligands within the IL-6Rβ (gp130). Both models assumed the peptide ligand was in an extended configuration. In an embodiment of the invention, the phosphotyrosine residue interacts with a positively charged pocket (pocket 1) within the SH2 domain formed by the side chains of K591 and R609. The +3 Q/C was predicted to interact with a hydrophilic pocket (pocket 2) formed by the side chains of E638, Y640 and Y657. In the Hemmann model, the phosphotyrosine was predicted to interact with the side chain of R609 (pocket 1) and the +3 Q with the side chains of Y657, C687, S691 and Q692 (pocket 2).

Stat3 proteins in which mutations were introduced to alter side chains from charged or polar to non-polar within amino acid residues were predicted in each model to be critical for Stat3 binding (FIG. 1B). The recombinant Stat3 proteins were expressed in Sf9 insect cells and purified to equivalent levels using Ni-NTA resin (FIG. 1C). Peptide affinity immunoblot studies using Stat3-3M to test the pocket 2 component of the Chakraborty model demonstrated levels of Stat3-3M bound to Y704 and Y744 phosphododecapeptides similar to wild type Stat3 (FIG. 6A). Peptide affinity immunoblot studies using Stat3-4M to test the pocket 2 component of the Hemmann model also demonstrated levels of binding of Stat3-4M bound to Y704 and Y744 phosphododecapeptides equivalent to wild type Stat3 (FIG. 6A). Furthermore, Stat3-6M, in which all six amino acid residues predicted by both models to form pocket 2 were mutated, bound both phosphododecapeptides at levels similar to wild type Stat3. These results do not support either model for Stat3 SH2 binding to +3 Q/C within phosphopeptide ligands.

To test the pocket 1 component of the two models and to ensure that our peptide pull-down system was sufficiently sensitive to detect reduced binding of Stat3 containing mutations in pocket 2, either K591L or R609L was added to the 3M mutant to generate Stat3-3M+K591L and Stat3-3M+R609L. Addition of either mutation resulted in elimination of binding to both Y704 and Y744 phosphododecapeptides indicating that each of the side chains of K591 and R609 contribute to binding of the phosphotyrosine.

To confirm these findings and to determine if introduction of the pocket 2 mutations resulted in subtle alterations in kinetics of binding undetectable using phosphopeptide affinity immunoblot analysis, mirror resonance affinity assays were performed using phosphorylated and non-phosphorylated Y704 and Y744 dodecapeptides (FIG. 6A and FIG. 6B and Table 5). Review of the real-time mirror resonance affinity curves (FIG. 6B and FIG. 6C) and kinetic analysis (Table 5) revealed low or undetectable binding of Stat3-3M+R609L and Stat3-3M+K591L, respectively, to Y704 and Y744 phosphododecapeptide confirming the results of peptide immunoblot analysis. The pocket 2 mutant Stat3 protein, Stat3-3M, demonstrated kₐₛₛ, k_{diss} and K_{D} values for binding to Y744 phosphododecapeptide indistinguishable from wild type Stat3 binding to this peptide confirming the peptide immunoblot analysis. The kinetic results of Stat3-3M binding to Y704 revealed a K_{D} of 1.21 μM, which was increased 72% compared to wild type Stat3 and attributable to a slower kₐₛₛ. These results indicate that Stat3 SH2 binding to the +3 C within Y744 does not require any of the side chains predicted in either of the proposed models while those side chains proposed in the Chakraborty model make a contribution, albeit small, to binding of Stat3 SH2 to +3 Q within Y704.

### Example 12

### Computational Modeling of Stat3 SH2 Binding to +3 Q within Y704 Phosphododecapeptide

Stat3 binds directly to the EGFR within regions of the receptor containing Y1068 and Y1086. The YxxQ motif is contained within both of these regions; each region also contains the consensus motif for Grb2 binding YxNx. The structure of the Y1068 phosphopentapeptide (EpYINQ) is available from its crystal structure bound by Grb2 (PDB code 1ZFP). The structure of Stat3 from W580 to L670 was obtained from the crystal structure of Stat3β homodimer bound to DNA (PDB code 1BG1). These structures were used too generate a new and more robust model for Stat3 SH2 binding to +3 Q/C by computational modeling of the interaction and identification of the interaction with the lowest energy. All energy minimization calculations were carried out under AMBER force field by using the DISCOVER/Insight II program. A total of 300 steps of conjugate gradient energy minimization were performed following rigid hand-docking to fit the pY of the EpYINQ peptide into the binding pocket comprised of residues K591 and R609 taking into consideration Van der Waals and Coulomb forces. The complex formed between Stat3-SH2 and EpYINQ with the lowest energy (FIG. 7A) had a total binding energy of −478.8 Kcal/mol. This computational result predicted that the major binding energy for this binding configuration comes from a hydrogen bond interaction involving oxygen within the +3 Q side chain and the peptide amide hydrogen at E638 located within a loop region of Stat3 SH2. Replacement of the EGFR pentapeptide EpYINQ with the G-CSFR Y704-based pentapeptide TpYVLQ did not change the length or angle of this hydrogen bond (FIG. 7B).

To test the contribution of the E638 amide hydrogen to binding to G-CSFR Y704 and Y744 phosphododecapeptide, Stat3-E638P was generated by site-directed mutagenesis, which eliminated the amide hydrogen donor predicted to bind with oxygen within the +3 Q side chain. Introduction into Stat3 of the E638P mutation did not alter secondary structure in computer modeling simulations or when recombinant protein was expressed and purified from Sf9 cells and examined directly by CD analysis. Peptide affinity immunoblot assays using recombinant Stat3-E638P (FIG. 1C) demonstrated no binding of Stat3-E638P to any of the G-CSFR derived peptides tested including Y704 and Y744 phosphododecapeptides (FIG. 6A); mirror resonance affinity studies (FIGS. 6B and C) confirmed these findings. These results strongly support an important role for the E638 amide hydrogen of Stat3 in binding of the +3 Q within Y704 phosphododecapeptide and the +3 C within Y744 phosphododecapeptide.

### Example 13

### The Side Chain of Amino Acid Residue R609 and the Amide Hydrogen of Residue E638 within the Stat3 SH2 Domain are Important for Binding and Activation of Stat3 by the Full-Length G-CSFR In Vivo

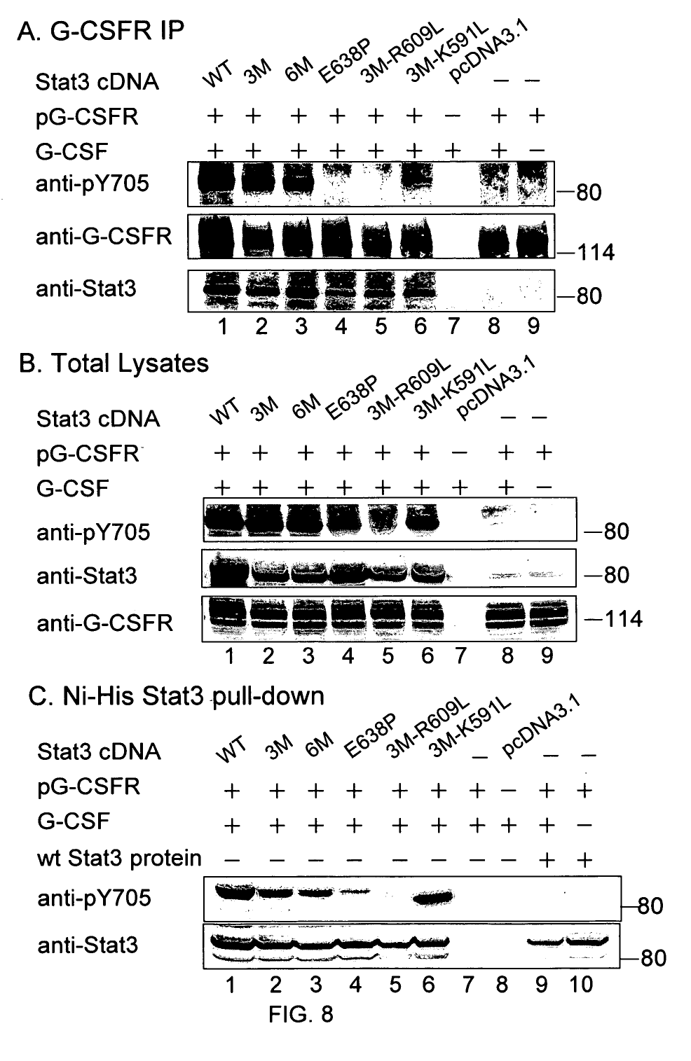

To determine if the side chains of amino acid residues K591 and R609 and the amide hydrogen of residue E638 within the Stat3 SH2 domain are important for binding of Stat3 to full-length G-CSFR, the inventors compared levels of wild-type and mutant Stat3 within immunoprecipitates of phosphorylated G-CSFR. G-CSFR was immunopreciptated from G-CSF-stimulated 293T cells co-transfected with full-length G-CSFR cDNA and either wild type or mutant Stat3 cDNA constructs (FIG. 8A). Equivalent levels of total and Y705-phosphorylated wild type Stat3, Stat3-3M and Stat3-6M protein were found within G-CSFR immunoprecipitates (lanes 1-3) as predicted from the peptide affinity results. In contrast, levels of total Stat3-E638P (lane 4), Stat3-3M-R609L (lane 5) and Stat3-3M-K591L present within G-CSFR immunoprecipitates were reduced by 40-50% compared to wild type Stat3. Of special note, levels of Y705-phosphorylated Stat3 (pStat3) proteins within G-CSFR immunoprecipitates were either undetectable (Stat3-E638P and Stat3-3M-R609L) or reduced 70-80% (Stat3-3M-K591L).

To determine the effects of reduced recruitment to the G-CSFR of the mutated Stat3 proteins on their activation, the inventors examined levels of pStat3 within the lysates of co-transfected cells (FIG. 8B) and following Ni-NTA agarose affinity purification of Stat3 (FIG. 8C). Levels of pStat3 were similar in lysates co-transfected with G-CSFR and wild type Stat3, Stat3-3M or Stat3-6M (lanes 1-3). In contrast, levels of pStat3 were reduced by 50% or more in cells transfected with Stat3-E638P (lane 4) and were almost completely absent in cells transfected with Stat3-3M-R609L (lane 5). In contrast, the level of pStat3 in cells transfected with Stat3-3M-K591L (lane 6) were reduced only slightly compared to pStat3 levels in cells transfected with Stat3-3M or wild type Stat3 (lanes 1 and 2). These findings confirm and extend the Y704 and Y744 phosphododecapeptide binding results and indicate that none of the residue side chains proposed previously by Chakraborty et al. or Hemmann et al. contribute to Stat3 recruitment and activation by the G-CSFR; rather, the side chain of R609 and the amide hydrogen of E638 make major contributions to Stat3 recruitment and activation by the G-CSFR in vivo while the side chain of K591 makes a minor contribution to these processes.

### Example 14

### Determine If Structural Mutations within the Stat3 SH2 Domain Results in a Switch in the IL-6 Response from Stat3-Dominant to Stat1-Dominant

Wild type Stat3 and Stat3 constructs containing mutations within the SH2 domain will be expressed in Stat3-deficient murine embryonic fibroblasts (MEFs). Cells will be examined before and after IL-6 stimulation for kinetics of Stat3 and Stat1 activation (Table 6) to assess if the switch from Stat3 to Stat1 in Stat3-deficient MEFs is maintained or lost. The wild-type MEF cell line was derived and immortalized from 14-day old embryos of Stat3 floxed/floxed mice; the Stat3-deficient MEFs were derived from the wild-type MEF cell line by infection with adenovirus expressing Cre recombinase followed by limiting dilution. The Stat3-deficient MEFs will be transfected with wild-type or mutant Stat3 cDNA constructs subcloned into pZeo using Fugene6 (Roche). After selection in zeocin (400 μg/ml), individual clones will be isolated and immunoblotted for level of Stat3 protein expression. Three-to-five clones from each transfection with levels of Stat3 expression equivalent to that in wild-type MEFs will be stimulated with IL-6 (200 ng/ml; R & D Systems) and sIL-6Rα (250 ng/ml; R & D Systems), and assessed for the kinetic- and dose-response of Stat3 and Stat1 activation by EMSA using hSIE duplex oligonucleotides and pStat3 and pStat1 specific antibodies, as described. Wild-type MEFs and Stat3-deficient MEFs clones derived from cells transfected with empty pZeo, selected in zeocin and stimulated with IL-6/sIL-6Rα will serve as controls.

IL-6/sIL-6Rα stimulation of wild-type MEFs will activate both Stat3 and Stat1 (Table 6) while stimulation of Stat3-deficient MEFs will demonstrate a switch that is reversed by forced expression of wild-type Stat3 as described previously. In contrast to forced expression of wild type Stat3, forced expression of Stat3-R609L or Stat3-E638P may not be able to reverse the switch, i.e. the switch will be maintained because of the reduced ability of these mutated Stat3 constructs to bind to Stat3 pY peptide ligands in vitro and be activated in vivo. Furthermore, forced expression of Stat3-K589L will not maintain the switch.

Stat3 activation by cytokine/growth factor activated receptors such as those for G-CSF is thought to occur through two pathways—one that requires receptor pY peptide motifs and one that does not. Results of G-CSFR and Stat3 co-expression studies in 293T cells indicate that Stat3 recruitment and activation downstream of G-CSF that occurs independently of G-CSFR pY motifs also requires that Stat3 be competent to bind to pYxxPolar recruitment sites. Consequently, if the results do not support the specific embodiment that a structural switch at the SH2 domain can be achieved even in G-CSFR-expressing MEFs, this would suggest that SH2-mutated Stat3 while unable to bind to pY peptide motifs may be recruited to the IL-6 or G-CSF receptor complex in MEFs through other intact domains, such as its coiled-coil domain. This non-SH2-mediated recruitment and activation may be sufficient to prevent a switch to a Stat1-predominant response. The coiled-coil domain was shown to contribute to Stat3 SH2 mediated binding to and activation by the EGFR and IL-6R in a series of mutational deletion studies although this contribution was subsequently shown to be indirect and mediated via interdomain interactions within Stat3.

These studies will support any embodiment related to a genetically based structural switch from Stat3 to Stat1 that can be achieved by impairing the ability of Stat3 SH2 to bind to its pY peptide ligands leaving endogenous wild type Stat1 unimpeded.

TABLE 6
Experiments to determine if a structural switch can be established
at the site of Stat3 recruitment to gp130 in MEFs

| x | | | Stat3 | Stat1 | Switch: |
|---|---|---|---|---|---|
| p | MEFs | Stat3 cDNA | activation | activation | Stat3 to 1 |
| 1 | WT | — | ++ | + | No |
| 2 | Stat3 −/− | — | − | +++ | Yes |
| 3 | Stat3 −/− | WT | ++ | + | No |
| 4 | Stat3 −/− | R609L | −/+ | +++ | Yes |
| 5 | Stat3 −/− | K589L | +/++ | + | No |
| 6 | Stat3 −/− | E638P | −/+ | +++ | Yes |
| 7 | Stat3 −/− | R609L + E638P | −/+ | + | Yes |
| 8 | Stat3 −/− | R609L + E638 + | − | + | Yes |
| | | K589L | | | |

### Example 15

### Determine If Compounds that Block Stat3 SH2-pY Peptide Interactions Yet Spares Stat1 SH2-pY Peptide Interactions can Serve as a Chemical Switch

Screening will be based on the existing model of the structure of Stat3 bound to EGFR Y1068 pY peptide ligand (FIGS. 2 and 3). The results of the experiments performed will confirm the virtual ligand screening approach. If the Stat3-deficient MEF clones containing the Stat3-E638P construct maintain the switch from Stat3 to Stat1, this would indicate that targeting the binding site for +3 Q (or polar residues C, S or T) within the Stat3 SH2 may be sufficient to generate a chemical switch. An exemplary strategy outlined below, which is similar to that recently employed by Huang N et al., will be performed. Huang et al., performed a virtual ligand screen of a 2 million compound virtual library targeting the site within Lck SH2 that binds the +3 I of the pY peptide (pYEEI) shown to be preferentially bound by this SH2 domain. Alternatively, if only Stat3-deficient MEF clones containing either the Stat3-3M-R609L+E638P or the Stat3-3M-R609L+E638P+K591L constructs maintain the switch from Stat3 to Stat1, this would indicate that both the pY phosphate binding site and the +3 polar binding site need to be targeted to generate a chemical switch, and a virtual ligand screen that includes both of these sites will be performed.

### Example 16

### Identification of Candidate Compounds that Block Stat3 SH2 Binding to pY Peptides Containing PYxxPolar Motifs While Sparing Stat1 SH2 Binding to its pY Peptide Ligands

Structure-based computational screening of a virtual chemical library is designed to identify novel compounds complementary to a putative binding site on an enzyme or receptor. This approach used as part of a structure-based drug design strategy has successfully contributed to the introduction of over 50 compounds into clinical trials including thrombin inhibitors, CD4 blockers, HIV integrase inhibitors and growth hormone antagonists, for example. If the results of the experiments outlined in EXP IIA1 indicate that elimination of the Stat3 E638 amide hydrogen is sufficient to maintain a switch from Stat3 to Stat1 in Stat3-deficient MEFs, it would be appropriate to apply virtual screening techniques followed by experimental assays to identify small molecular weight (MW) nonpeptidic compounds targeting the +3 Q binding site, as described for the +3 I binding site of Lck.

Models of the 3D structures of the Stat3 and Stat1 SH2 domains bound to their respective pY peptide ligands have been generated. These models will be used in a structure-based virtual ligand screening approach to determine if candidate compounds that block Stat3 SH2-pY peptide interactions while sparing Stat1 SH2-pY peptide interactions can be identified. This screen will be performed using the Tripos software suite. The +3 Q binding site consists, at this point in the model development, of the E638 amide hydrogen, which forms a hydrogen bond with the +3Q oxygen—as its major determinant. The remainder of the binding pocket consists of a hydrophobic pocket composed of the non-polar atoms within the side chains of V637, E638 and Y640.

### Example 17

### Generation of the Virtual Compound Library

A 3D database of 2.7 M commercially available compounds will be built as described. Briefly, the 1D/2D structures of the compounds will be obtained from 23 compound suppliers, as described; 1D/2D structures will be converted to 3D as described which involves database file format conversion, initial 3D geometry generation, hydrogen addition, charge assignment, and force field optimization using SYBYL.

### Example 18

### Identification of Candidate Compounds that Bind to the +3 Q Binding Site in the Stat3 SH2 While Sparing the +2 K Binding Site within Stat1 SH2

The following describes an exemplary disclosure for identification of candidate compounds of the invention. The coordinates of the most up-to-date 3D structure of the Stat3 SH2 domain bound to the pY peptide pYINQ with all water molecules removed will serve as the starting point for docking and subsequent calculations. Charges and hydrogens are added to the protein by SYBYL. Docking will target the +3 Q binding site of the Stat3 SH2 domain described herein. All docking calculations are carried out with DOCK using flexible ligands and rigid receptor based on the anchored search method. The solvent-accessible surface is calculated with the program DMS31 using a probe radius of 1.4 Å. Sphere sets, required for initial placement of the ligand during database screening, are calculated with the DOCK associated program SPHGEN. Only those spheres within 6 Å of the pY +3 binding site and within 3 Å of the structurally determined location of the +3 Q residue will be selected for the search. Ligand-receptor interaction energies are approximated by the sum of electrostatic and van der Waals components as calculated by the GRID method. To avoid identifying compounds that bind to the pY binding site, phenolphosphate will be maintained in this site, as described and the rest of the peptide ligand deleted. To avoid improper electrostatic interactions between docked ligands with this added moiety, a total charge of zero is assigned to it.

Database screening will initially select compounds that contain 10 or less rotatable bonds and between 10 and 40 non-hydrogen atoms. Ligand flexibility is considered by dividing each compound into a collection of non-overlapping rigid segments. Individual rigid segments with five or more heavy atoms (e.g., aromatic rings) are selected as “anchors”. Each anchor is docked separately into the binding site in 200 different orientations, based on different overlap of the anchor atoms with the sphere set, and is energy-minimized. The remainder of each molecule is built onto the anchor in a stepwise fashion until the entire molecule is built, with each step corresponding to a rotatable bond. At each step, the dihedral about the rotatable bond, which is connecting the new segment to the previously constructed portion of the molecule, is sampled in 10 increments and the lowest energy conformation selected. During the build-up procedure, selected conformers are removed on the basis of energetic considerations and maximization of diversity of the conformations being sampled and the orientation with the most favorable interaction energy will be selected. To avoid bias toward the selection of high molecular weight compounds because of the contribution of the compound size to the energy score, the energy score will be normalized by the number of heavy atoms N. This will allow the selection of smaller MW compounds with the best complementarities to the +3 Q binding site, better absorption and disposition properties and they will be better suited for later lead optimization efforts. From this procedure, a total of 25,000 compounds will be selected based on N^{1/2} normalized van der Waals attraction interaction energy. These compounds will be screened for lack of interaction with Stat1 SH2 using the most validated version of our current model. Compounds that score low in their ability to interact with the +2 K binding site will be subjected to secondary screening for binding the +3 Q binding site of Stat3 SH2 performed by applying a more rigorous docking method that includes simultaneous energy minimization of the anchor fragment during the iterative build-up procedure. Two sets of 1,000 compounds will be selected on the basis of the total interaction energy and the N^{1/2} normalized total interaction energy scores. To facilitate the selection of chemically diverse compounds for biologically assay, structural clustering will be applied. This is performed by dividing each set of 1000 compounds from the secondary dock run into chemically dissimilar clusters by applying the Tanimoto similarity indexes using the program MOE. Compounds for biological assay are selected from the dissimilar sets performed by individually analyzing the clusters and selecting compounds from each cluster based on several criteria such as adequate solubility (ClogP≦5), molecular weight (500 Da), the number of the hydrogen bond donors and acceptors (≦10), and chemical stability.

### Example 19

### Exemplary Acquisition and Testing of Candidate Compounds

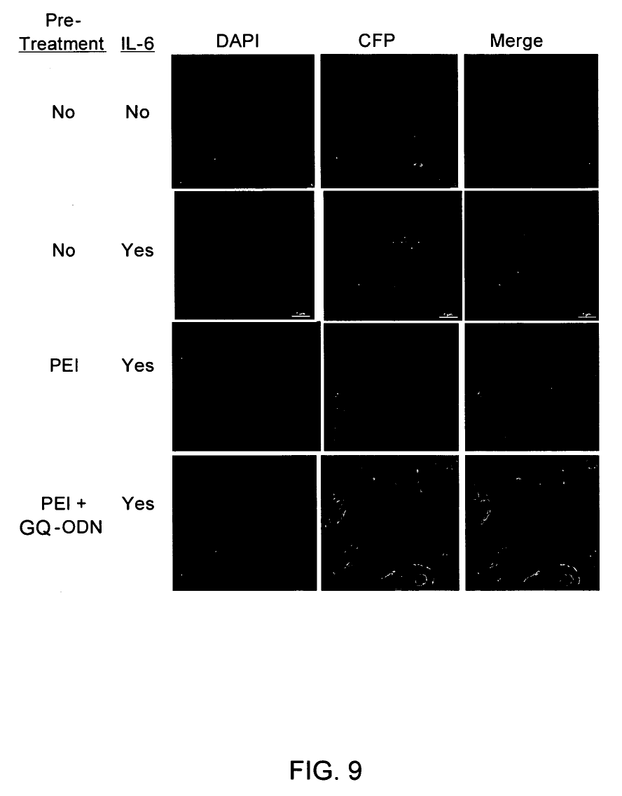

Candidate compounds will be purchased from their manufacturer and assayed first by fluorescent microscopy for ability to block nuclear translocation of Stat3 within IL-6-stimulated HepG2 cells transiently transfected with cyan fluorescent protein (CFP)-tagged Stat3. CFP-Stat3 has been shown by us to become phosphorylated on Y705, dimerize and bind to duplex DNA (see FIG. 9), and this screen would have identified the GQ-ODN T40214. Briefly, cells are grown on cover slips and transiently transfected with CFP-Stat3 contained within the pECFP vector (Clontech), as described. A 10 mM stock solution of each compound is prepared in DMSO. Two days later, cells are incubated for 2 hours in 24-well plates with medium containing 100, 10, 1, 0.1 μM compound concentrations. A two-hour pre-treatment with compound is based on previous studies examining the effects of agonists and antagonists on the nuclear distribution of nuclear hormone receptors. Following pre-incubation with compounds, cells will be stimulated with IL-6 (25 ng/ml) for 30 minutes, as described, fixed in 4% formaldehyde (pure, EM-grade, Polysciences Inc) for 30 min in 0.1 M Pipes, pH, 7.4, then specifically stained for DNA with DAPI stained (1 g/ml, 30 min, to achieve intense nuclear labeling) and examined by fluorescence microscopy. Each experiment will contain at least three controls: 1) cells not stimulated with IL-6, 2) cells pre-treated with the highest concentration of DMSO and stimulated with IL-6 and 3) cells pre-treated with PEI+GQ-ODN (T40214) and stimulated with IL-6.

Compounds that score positive in this screen will be evaluated for non-specific cell toxicity using trypan blue exclusion, as described. Non-toxic, positively scoring compounds will undergo more extensive kinetic evaluation in the nuclear translocation assay. Briefly, compounds scoring positive will be incubated with CFP-Stat3-transfected cells at their most potent concentration judging from the initial screen but for various times before stimulation with IL-6 (0, 1, 3, 10, 30, 60 min and 3 and 6 hr), fixation and examination by fluorescence microscopy to identify the optimum and minimum period of exposure required. Once the optimum incubation time is identified, compound will be exposed to CFP-Stat3 transfected cells for the optimum time but over a refined range of concentrations with the concentrations tested depending on the initial screening results. Positive compounds will be examined by EMSA for the ability to destabilize Stat3 dimers in vitro, as described and to inhibit Stat3 binding in EGFR Y1068 pY peptide affinity immunoblot assays performed as described. This will begin to establish that they act as predicted by interfering at the +3 Q binding site within the Stat3 SH2.

To further validate that the active compounds that destabilize Stat3 dimers and inhibit EGFR Y1069 phosphopeptide binding are doing so by binding directly to the SH2 domain, fluorescence titration experiments will be undertaken as described. These experiments take advantage of the presence of a tryptophan in the Linker-SH2 protein at W623. By use of an excitation wavelength of 270 nm, an emission maximum is obtained at 585 nm; this wavelength is monitored upon addition of candidate compounds that inhibit EGFR Y1069 binding to Stat3. Addition of the compounds to the Stat3 Linker-SH2 domain will quench fluorescence upon binding. Double reciprocal plots will be calculated to determine the dissociation constants of those compounds that quench W623 fluorescence. A compound that does not inhibit binding of Stat3 to EGFR Y1068 PDP will be included as a negative control in these studies.

Selectivity of candidates for Stat3 vs. Stat1 will be confirmed in vitro by EMSA in Stat1 homodimer destabilization assays and in vivo by EMSA using HepG2 cells, as described. Briefly, compound will be added to HepG2 cells at an early plateau concentration based on the dose response in the nuclear translocation assay. After incubation for 2 hours or for the optimum pre-treatment period determined above, the cells are incubated without or with IL-6 (25 ng/mL) or IFN-γ (1,000 U/ml) at 37° C. for 30 minutes before extraction and analysis by electrophoretic mobility shift assay (EMSA) using hSIE duplex oligonucleotides. Candidates that demonstrate specificity for Stat3 in these assays will be tested in Stat3-deficient MEF cell clones reconstituted with wild type Stat3 for the ability to prevent wild type Stat3 reversal of the switch from Stat3 to Stat1 following IL-6 activation, as described.

Candidates that show evidence of good activity against Stat3 and selectivity for Stat3 vs. Stat1 will be used as a lead compound for the synthesis of small chemical library. The library will be screened and compared with the parent compound for improved activity and selectivity and ability to serve as chemical switches as outlined above.

The chemical screen will identify several novel compounds that inhibit Stat3 nuclear translocation, destabilize Stat3 dimers, inhibit Stat3 binding to EGFR Y1068 phosphopeptide and bind directly to Stat3 Linker-SH2 protein. As support for this approach, see the recent results of Huang et al., 2004. Using virtual ligand screening and a similar strategy, Huang et al. successfully identified a total of 288 unique compounds within a 2,000,000 compound virtual library predicted to bind to the +3 Ile binding site within the Lck SH2 domain (hit rate of 0.014%). Of the 288 candidates identified, a total of 196 were available from commercial vendors. Thirty-four of 196 were shown to inhibit Lck SH2 domain association with ITAM2 phosphotyrosine peptide. Thirteen of 34 demonstrated inhibitory activity in mixed lymphocyte culture assays. In the case of four of these 13, fluorescence titration experiments supported the conclusion that they bound the Lck SH2 domain. The screen will add the additional restriction that candidate compounds identified in the first round of computational screening do not bind to the Stat1 SH2 binding site for the pY peptide +2 K. This will reduce the number of “hits.” However, given the distinct features of the Stat1 SH2 binding site for the +2 residue, i.e. a salt bridge between the +2 K and the side chain of E632, this reduction should not be very large. Assuming reduction in the initial “hit” rate from 0.014 achieved by Huang et al to 0.010%, these would result in the identification of 200 unique compounds.

### Example 20

### Virtual Ligand Screening (VLS) to Identify within Compound Libraries Candidates Capable of Specifically Blocking the Phosphotyrosine-Binding Site within the Stat3 SH2 Domain

The following exemplary protocol used for virtual ligand screening (VLS) comprises four steps: 1) preparation of the Stat3-SH2 structure for computer docking studies; 2) selection and conversion of the virtual compound library database files; 3) computer docking of compounds onto the Stat3 SH2-pY ligand binding pocket; and 4) ranking and editing candidates for purchase and further biochemical testing.

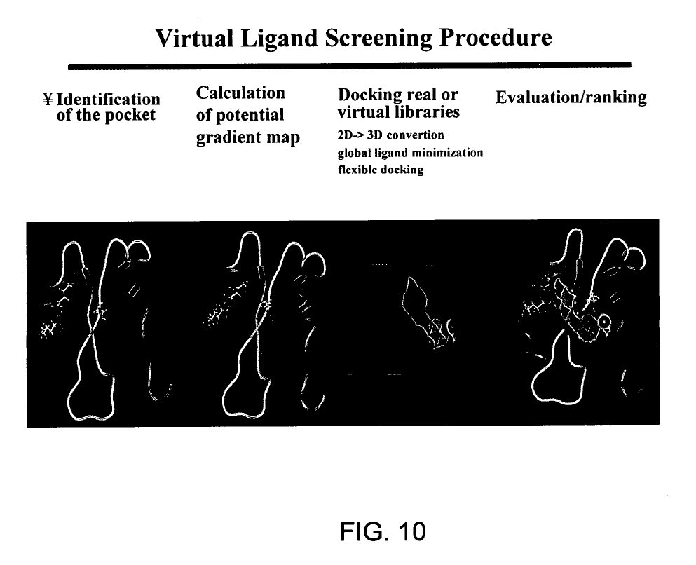

Recruitment of Stat3 to tyrosine phosphorylated receptors, including the epidermal growth factor receptor (EGFR), the granulocyte colony-stimulating factor receptor (G-CSFR) and the interleukin (IL) 6 receptor, is mediated by an interaction between specific pY residues within the receptor and the Stat3 SH2-pY peptide binding site (Shao et al. 2004; Shao et al., in press). The Stat3 SH2 pY binding site consists of two subsites—one is a general binding site (GBS in Panel A of FIG. 10) and the other is the specific binding site (SBS in Panel A of FIG. 10). The general binding site shares features with most other SH2 domains and is comprised in the Stat3 SH2 domain of the side chains of R609, which makes the major contribution, and K591, which makes a minor contribution (Shao et al., in press). The central feature of the specific binding site is the amide hydrogen within the peptide backbone of the Stat3 SH2 domain at residue E638, which serves as a hydrogen bond donor (Shao et al. 2004; Shao et al., in press).

Preparation of the Stat3-SH2 structure for computer docking studies. To prepare the Stat3 SH2 domain for computer docking, the inventors isolated the three-dimensional structure of the SH2 dimers from the total structure of Stat3 homodimers bound to DNA deposited in the PDB databank (PDB code 1BG1) and converted is to an ICM-compatible file by adding hydrogen atoms, modifying unusual amino acids, making charges adjustment and performing additional cleanup.

Selection and conversion of the virtual compound library database files. Commercial chemical databases were selected as sources of compounds for in silico screening. Chemical databases were obtained from ChemBridge, Asinex, ChemDiv, Enamine, KeyOrganics and LifeChemicals. In the aggregated, these compound databases contained over one million chemically feasible, drug-like compounds. Before screening, each compound pools was converted from an sdf file to an index file or inx file to make it compatible for screening within the ICM program.

Computer docking of compounds onto the Stat3 SH2-pY ligand-binding pocket. The inventors selected the amide hydrogen of E638 as the central point of the binding pocket, which consisted of a cube with the dimensions 16.0×16.9×13.7 angstrom. A series of grid potentials within the binding pocket are calculated which describe the interaction of flexible ligands and receptor maps are constructed as depicted in the FIG. 10 (second panel). Monte Carlo simulation is used to randomly assign all possible conformations of interaction of the ligand within the binding pocket. A flexible docking calculation is performed in order to find the global minimum energy score, which predicts the optimum conformation of the compound within the pocket (third panel in FIG. 10).

Ranking and editing candidates for purchase and further biochemical testing. After docking simulation of each compound within a pool to the binding pocket is completed, the results of a list of compounds and their global minimal energy score are reviewed. Compounds are selected for purchase and biochemical testing if their global minimum energy score is ≦−30 and they form a hydrogen bond with amide hydrogen of E638 interact within the binding pocket is such a way as to restrict access to the amide hydrogen.

### Example 21

### Stat3 Y705 Phosphorylation Inhibition Assay

Human hepatoma cells (HepG2) are grown in 6-well plates to confluence. Each candidate compound is dissolved in DMSO to achieve a concentration of 10 mM immediately before testing. Candidate compound dissolved in DMSO or an equivalent volume of DMSO alone is added to a test well of HepG2 to achieve a concentration of 100-to-200 μM (100 μM was used for candidate compounds 1 through 23; 200 μM for all subsequent candidate compounds). After incubation at 37° C. for 1 hr, cells are stimulated with IL-6 (30 ng/ml) for 30 min at 37° C. The medium is removed and 200 μl of high-salt extract buffer (20 mM HEPES, pH 7.9, 20 mM NaF, 1 mM Na₃VO₄, 1 mM Na4 P₂O₇, 1 mM EDTA, 1 mM EGTA, 1 mM DTT, 420 mM NaCl and 20% glycerol) is added and incubated at 95° C. for 5 min. Extracts are harvested and 20 μl separated by SDS-PAGE, blotted onto membrane and developed with murine monoclonal antibody against Stat3 phosphotyrosine 705 (3E2; Cell Signaling Technology Inc, Beverly, Mass., USA). Candidate compounds that inhibit IL-6-activated Stat3 phosphorylation are retested and if the result is reproduced tested over a range of concentrations to establish an IC₅₀.

### Example 22

### Exemplary Screening Results

The present inventors have performed an in silico screen of 400,000 compounds from multiple chemical companies, including ChemBridge Corporation (San Diego, Calif.), Asinex Ltd. (Moscow, Russia), Enamine Ltd. (Kiev, Ukraine), Key Organics Ltd. (Camelford, UK), and Life Chemicals, Inc. (Burlington, Ontario), for example. They have identified approximately 100 compounds that met criterion for purchasing and testing from these exemplary companies and have tested approximately 80 for the ability to inhibit ligand-stimulated phosphorylation. One exemplary compound (compound 3 in Table 7) inhibited Stat3 phosphorylation with an IC₅₀ of 100 micromolar and was not toxic to cells. The chemical formula of this compound is listed in the table, and the structure is shown in FIG. 10.

TABLE 7
Exemplary Candidate Compounds of the Invention

| # of compounds in | | | MW | | Mg | Stat3 pY |
|---|---|---|---|---|---|---|
| SCRN | # in SCRN | Formula | (Da) | Score | order | Test |
| 10000 | 1 | C30H16N2O11 | 580.46 | −41.16 | 5 | no |
| | 2 | C17H11Cl2NO4S | 396.24 | −36.79 | 10 | no |
| 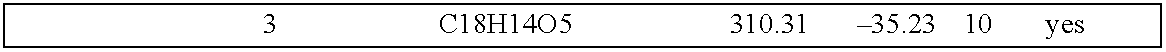 | | | | | | |
| | 4 | C11H1ON2O4 | 234.21 | −34.85 | 10 | no |
| | 5 | C30H24N2O10 | 572.53 | −34.11 | 10 | no |
| | 6 | C20H15N5O3S | 405.43 | −33.79 | 10 | no |
| | 7 | C21H12N2O7 | 404.33 | −33.74 | 10 | no |
| | 8 | C14H9NO7 | 303.23 | −33.01 | 10 | no |
| | 9 | C11H8O5 | 220.18 | −32.47 | 10 | no |
| | 10 | C17H1OBrNO5 | 388.17 | −32.39 | 5 | no |
| | 11 | C10H6O5 | 206.15 | −32.34 | 10 | no |
| | 12 | C20H17NO5 | 351.36 | −31.98 | 10 | no |
| | 13 | C26H20N2O8 | 488.45 | −31.44 | 10 | no |
| | 14 | C24H20N2O4 | 400.43 | −31.44 | 10 | no |
| | 15 | C12H13NO5 | 251.24 | −31.41 | 10 | no |
| | 16 | C17H11NO6 | 325.28 | −31.41 | 10 | no |
| | 17 | C15H8BrNO4 | 346.14 | −31.37 | 10 | no |
| | 18 | C12H10O4 | 218.21 | −31.31 | 10 | no |
| | 19 | C23H14N2O7 | 430.37 | −31.31 | 10 | |
| | 20 | C18H17NO7 | 359.33 | −31.25 | 5 | no |
| | 21 | C24H16N2O10 | 492.4 | −30.91 | 10 | No |
| 50000 | 22 | C21H17BrN2O4S2 | 505.4 | −38.95 | 10 | No |
| | 23 | C30H19N3O8 | 549.5 | −38.1 | 10 | No |
| | 24 | C23H17N3O6 | 431.4 | −37.95 | 10 | no |
| | 25 | C21H13NO5S | 391.4 | −37.53 | 10 | no |
| | 26 | C17H11Cl2NO4S | 396.24 | −36.53 | 10 | |
| | 27 | C19H14N2O5S | 382.39 | −36.02 | 10 | no |
| | 28 | C17H15N3O3 | 309.32 | −35.14 | 5 | No |
| | 29 | C10H10N2O7S | 302.26 | −35.14 | 5 | no |
| | 30 | C17H13NO4S2 | 359.41 | −34.69 | 10 | |
| | 31 | C16H16N2O4 | 300.31 | −34.51 | 10 | no |
| | 32 | C18H17NO6 | 343.34 | −34.44 | 10 | no |
| | 33 | C24H24N2O5S2 | 484.58 | −34.32 | 10 | no |
| | 34 | C20H14N2O2S | 346.4 | −34.2 | 5 | no |
| | 35 | C21H12N2O7 | 404.33 | −34.09 | | |
| | 36 | C19H15IN2O5 | 478.24 | −34.09 | 5 | no |
| 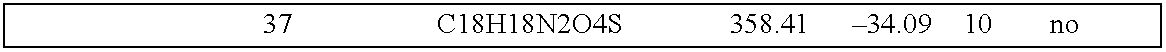 | | | | | | |
| | 38 | C18H13NO4S2 | 371.43 | −34.08 | 10 | no |
| | 39 | C17H11NO6 | 325.28 | −33.74 | 10 | no |
| | 40 | C23H17N3O6 | 431.4 | −33.72 | 10 | no |
| | 41 | C20H16N2O7 | 396.36 | −33.53 | 10 | no |
| | 42 | C19H14N2O5S | 382.39 | −33.43 | 10 | no |
| | 43 | C21H22N2O4 | 366.42 | −33.22 | 10 | no |
| | 44 | C16H15NO5 | 301.3 | −32.88 | 10 | no |
| | 45 | C17H11ClN4O5 | 386.75 | −32.79 | 10 | no |
| | 46 | C21H14F2N2O4 | 396.35 | −32.32 | 10 | no |
| | 47 | C25H15N3O7 | 469.41 | −32.22 | 10 | no |
| | 48 | C11H13NO4 | 223.23 | −31.92 | 10 | no |
| 20000 | 50 | | 39.05 | | | |
| | 51 | | 33.56 | | | |
| | 52 | | 33.46 | | | |
| | 53 | | 29.23 | | | |
| | 54 | | | | | |
| | | | 28.44 | | | |
| | 55 | | 27.28 | | | |
| 20000 | 57 | C45H42N2O6 | 706.84 | −38.25 | 5 | no |
| | 58 | C41H26O5 | 598.66 | −38.41 | 10 | no |
| | 59 | C29H17NO8 | 507.46 | −35.41 | 10 | no |
| | 60 | | 31.63 | | | |
| | 61 | | 30.81 | | | |
| | 62 | C24H12N6O9 | 528.39 | −33.33 | 10 | no |
| 50000 | 63 | C20H15N2O4S2 | 411 | −39.57 | 10 | no |
| 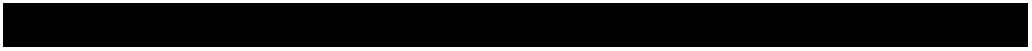 | | | | | | |
| | 65 | C12H10NO6 | 264 | −33.29 | 10 | No |
| | 66 | C26H23N3O7S2 | 551 | −31.88 | 10 | No |
| | 67 | C24H19N5O2S2 | 473 | −31.78 | 10 | No |
| | 68 | C20H12NO5 | 346 | −30.93 | 10 | no |
| | 69 | C15H11O4 | 255 | −30.14 | | No |
| | 70 | C19H11N2O6Br2 | 523 | −33.17 | 10 | No |
| | 71 | C24H19N4O6S2FCl2 | 613 | −33.05 | 10 | no |
| 40000 | 75 | | | −30.8 | | No |
| | 76 | | | −30.49 | | |
| | 77 | | | −30.22 | | |
| 10197 | 78 | | | −37.69 | | |
| | 79 | | | −37.05 | | |
| 65000 | 80 | C19H15N2O4S3 | 431 | −38.27 | | |
| | 81 | C13H16NO6S4 | 410 | −35.71 | 5 | |
| | 82 | C13H13NO6S | 311 | −35.5 | 5 | no |
| | 83 | C2OH14N2O5S2F | 444 | −35.48 | 5 | no |
| | 84 | C15H13NO5S | 319 | −35.3 | 5 | |
| | 85 | C18H12NO6 | 338 | −34.86 | 5 | no |
| | 86 | C19H15N2O5S2 | 415 | −34.7 | 5 | no |
| | 87 | C23H19N2O5S | 435 | −34.03 | 5 | no |
| | 88 | C18H14N3O5S | 384 | −32.7 | | |
| | 89 | C15H18NO5S | 324 | | 5 | no |
| | | | | −32.69 | | |
| | 90 | C12H14NO5S | 284 | −32.36 | 5 | No |
| | 91 | C12HISNO5S | 285 | −32.36 | 2 | No |
| | 92 | C12H13N2O6S | 349 | −31.8 | 5 | No |
| | 93 | | | −31.56 | | |
| | 94 | | | −31.02 | | |
| | 95 | C14H12NO5S | 306 | −30.66 | 5 | No |
| | 96 | | | −30.08 | | |
| | 97 | C23H19N2O5S2 | 467 | −30 | 5 | No |
| | 98 | C21H17N2O7S2 | 578 | −32.88 | 5 | no |
| | 99 | C25H21N4O5S2 | 521 | −32.11 | 5 | no |
| 25000 | 101 | | | −37.05 | | |
| | 102 | | | −32.85 | | |
| | 103 | | | −32.55 | | |
| | 104 | | | −32.07 | | |
| 20000 | 105 | | | −32.87 | | |
| 20000 | 106 | | | −33.17 | | |
| 46913 | 107 | | | −35.9 | | |
| | 108 | | | −35.38 | | |
| | 109 | | | −34.57 | | |
| | 110 | | | −33.95 | | |
| | 111 | | | −33.18 | | |
| 50000 | 112 | | | −50 | | |
| | 114 | | | −38.96 | | |
| | 115 | | | −38.54 | | |
| | 116 | | | −37.11 | | |
| | 117 | | | −37.03 | | |
| | 118 | | | −35.87 | | |
| | 119 | | | −35.79 | | |
| | 120 | | | −34.28 | | |
| | 121 | | | −34.23 | | |
| | 122 | | | | | |
| | | | | −33.73 | | |
| | 123 | | | −33.69 | | |
| | 124 | | | −33.58 | | |
| | 125 | | | −33.44 | | |
| | 126 | | | −33.34 | | |
| | 127 | | | −33.23 | | |
| | 128 | | | −33.09 | | |
| | 129 | | | −32.61 | | |
| | 130 | | | −32.26 | | |
| | 131 | | | −31.8 | | |
| | 132 | | | −31.73 | | |
| | 133 | | | −31.65 | | |
| | 134 | | | −31.6 | | |
| | 135 | | | −31.51 | | |
| | 136 | | | −30.31 | | |
| | 137 | | | −30.31 | | |
| | 138 | | | −30.21 | | |

The structure of exemplary compound 3 in Table 7 is provided in FIG. 11. In additional embodiments of the invention, the exemplary compound 3 was then used to screen the commercially available ChemBridge library to extract from it compounds with similar structure. Of those compounds that emerged from this screen, nine not only were structurally similar to compound 3 but also met the exemplary criteria of binding to or blocking binding to the specific binding site at E638FIG. FIG. 12 provides the structures of these additional exemplary compounds of the invention.

### Example 23

### Exemplary Modifications to Compounds of the Invention

This example describes exemplary strategies that could be employed to alter potential or known Stat3 inhibitors, such as to improve activity of (one or more of) a lead compound and/or a derivative thereof and/or to minimize toxicity of a compound, and/or to improve the pharmacokinetic or pharmacodynamic properties of the lead compound, and/or to increase half-life and/or to reduce degradation, for example.

Virtual ligand screening identified exemplary compound 3 in Table 7, as described above. This specific compound has consistently tested positive for the ability to inhibit IL-6-mediated Stat3 tyrosine phosphorylation demonstrating an IC₅₀ of approximately 100 μM. Three exemplary strategies may be employed to optimize the Stat3 binding affinity and bioavailability of compound 3 and other lead compounds that may be identified by methods described herein or that are otherwise suitable. One or more of these strategies may employ a compound's structure and/or chemical property or properties. The first strategy will employ using the lead compound to screen a commercially available database, for example, for other drugs with similar structure; an exemplary database would include the ChemBridge Corporation database, which contains 683,740 drug-like compounds. The inventors have performed this screen with compound 3 and identified 2,302 compounds that show a high degree of similarity in configuration and chemical properties. Each of these 2,302 compounds was then docked into the Stat3 SH2 binding site, which yielded 24 compounds with scores of −30 or less, suggesting favorable energetics of interaction. Further analysis of these 24 compounds revealed 9 compounds that fulfilled exemplary requirements that they directly interact with the amide hydrogen of E638 (or, alternatively, the carboxylic oxygen of S636). Each of these compounds will be obtained and tested for the ability to inhibit IL-6-mediated Stat3 tyrosine phosphorylation.

A second exemplary strategy to optimize lead compound activity and bioavailability, as well as to minimize toxicity, is to use the structure of the lead compound to screen a database of drugs currently in the marketplace. Compounds that are identified in this screen will undergo an identical series of confirmatory studies as outlined above.

A third strategy the inventors can pursue is to perform structure-based chemical modifications of the lead compounds and test each resultant modification for inhibitory activity. Compound 3 can be separated into three functional groups—a carboxylic group at its head, a long carbonic chain serving as a linker, and a indole derivative group at its tail (see FIG. 11). Based on computational simulations, the inventors found that the carboxylic head group interacts with the general binding site of Stat3 SH2, blocking the potential interaction of this site with the phosphorylated tyrosine group of the pYXXQ/C/T motif within the activated receptor or Stat3 dimerization partner. One modification of a general type is to substitute a phosphorylated group for the carboxylic group in order to increase its polarization and charge interactions in this region. Based on the inventors' in silico model, one of the oxygens in the double-ring group within the tail end of compound 3 forms a hydrogen bond with the amide hydrogen of E638 of Stat3, which forms the core of the specific binding site of the Stat3 SH2 domain. The inventors will chemically modify this region to increase its binding affinity. One such modification is to replace the two oxygens with fluorine atoms. The carbonic chain linker region within the middle of compound 3 serves to connect the two functional end groups and to place each of them in the right position to interact with the general and specific Stat3 SH2 binding site. This region of compound 3 will be optimized to maintain the spacing of the two ends (9.9 angstroms) and to optimize the angle of interaction of each of the ends with its binding site.

These three strategies, alone and/or in combination, will permit the inventors to proceed to establish a relationship between the activity of the lead compound(s) and its derivatives. Establishing a quantitative structure-activity relationship method (QSAR) is useful for optimization of drug activity and before proceeding with in vivo testing. After similarity screening, the inventors can switch the in silico-modeling platform from ICM to SYBYL and perform ligand-based drug design with SYBYL modules, QSAR with CoMFA and VolSurf. Ligand-based drug design uses information about one or several well-known ligands as a basis for the optimization of lead compounds, which includes structure-activity relationship modeling (QSAR) and ADME predictions. QSAR can establish a relationship between a molecule's chemical properties and/or biological activity and its structure in order to design compounds with increased effectiveness. Based on the chemical properties of compound 3 and its derivatives, the inventors will build statistical and graphical models of activity from their structures and use these models to organize the structures and their associated data into molecular descriptors. These descriptors will be used to perform sophisticated statistical analyses that will reveal patterns in structure-activity data. Optimized ligands will be identified according to this structure-activity relationship and synthesized for testing.

Similarly, the inventors will predict and analyze a variety of absorption, distribution, metabolism and excretion (ADME) properties of compound 3 and its derivatives using VolSurf software. Three dimensional molecular interaction fields will be calculated and converted into simple molecular descriptors. These descriptors will quantitatively characterize the sizes, shapes, polarities, and hydrophobicities of compound 3 and its derivatives, which will be useful in generating predictive ADME models of compounds 3 and its derivatives. The inventors will use these predictions to eliminate from consideration for in vivo testing compounds identified in the above analysis with high toxicities.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Akira, S., Nishio, Y., Inoue, M., Wang, X. J., Wei, S., Matsusaka, T., Yoshida, K., Sudo, T., Naruto, M., and Kishimoto, T. (1994) Cell 77, 63-71
Batzer, A. G., Rotin, D., Urena, J. M., Skolnik, E. Y., and Schlessinger, J. (1994) Mol Cell Biol 14, 5192-5201
Becker, S., Groner, B., and Muller, C. W. (1998) Nature 394, 145-151
Bowman, T., Garcia, R., Turkson, J., and Jove, R. (2000) Oncogene 19, 2474-2488
Bromberg, J. F., Horvath, C. M., Besser, D., Lathem, W. W., and Darnell, J. E., Jr. (1998) Mol Cell Biol 18, 2553-2558
Bromberg, J. F., Wrzeszczynska, M. H., Devgan, G., Zhao, Y., Pestell, R. G., Albanese, C., and Darnell, J. E., Jr. (1999) Cell 98, 295-303
Caldenhoven, E., van, D. T. B., Solari, R., Armstrong, J., Raaijmakers, J. A. M., Lammers, J. W. J., Koenderman, L., and de, G. R. P. (1996) Journal of Biological Chemistry 271, 13221-13227
Cantley, L. C., and Songyang, Z. (1994) Journal of Cell Science Supplement 18, 121-126
Chakraborty, A., Dyer, K. F., Cascio, M., Mietzner, T. A., and Tweardy, D. J. (1999) Blood 93, 15-24
Chattopadhyay, A., Vecchi, M., Ji, Q., Mernaugh, R., and Carpenter, G. (1999) J Biol Chem 274, 26091-26097
Downward, J., Parker, P., and Waterfield, M. D. (1984) Nature 311, 483-485
Grandis, J. R., Drenning, S. D., Chakraborty, A., Zhou, M. Y., Zeng, Q., Pitt, A. S., and Tweardy, D. J. (1998) J Clin Invest 102, 1385-1392
Grandis, J. R., and Tweardy, D. J. (1993) Cancer Research 53, 3579-3584
Guruprasad, K., and Rajkumar, S. (2000) J Biosci 25, 143-156
Hemmann, U., Gerhartz, C., Heesel, B., Sasse, J., Kurapkat, G., Grotzinger, J., Wollmer, A., Zhong, Z., Darnell, J. E., Jr., Graeve, L., Heinrich, P. C., and Horn, F. (1996) Journal of Biological Chemistry 271, 12999-13007
Huang et al., J Med Chem. 2004 Jul. 1;47(14):3502-11.
Keilhack, H., Tenev, T., Nyakatura, E., Godovac-Zimmermann, J., Nielsen, L., Seedorf, K., and Bohmer, F. D. (1998) J Biol Chem 273, 24839-24846
Kuriyan, J., and Cowburn, D. (1997) Annu Rev Biophys Biomol Struct 26, 259-288
Leatherbarrow, R. J. (1998) GraFit Version 4, Erithacus Software Ltd., Staines
Margolis, B., Li, N., Koch, A., Mohammadi, M., Hurwitz, D. R., Zilberstein, A., Ullrich, A., Pawson, T., and Schlessinger, J. (1990) Embo J 9, 4375-4380
Munoz, V., and Serrano, L. (1997) Biopolymers 41, 495-509
Ogura, K., Tsuchiya, S., Terasawa, H., Yuzawa, S., Hatanaka, H., Mandiyan, V., Schlessinger, J., and Inagaki, F. (1999) J Mol Biol 289, 439-445
Okabayashi, Y., Kido, Y., Okutani, T., Sugimoto, Y., Sakaguchi, K., and Kasuga, M. (1994) J Biol Chem 269, 18674-18678
Okutani, T., Okabayashi, Y., Kido, Y., Sugimoto, Y., Sakaguchi, K., Matuoka, K., Takenawa, T., and Kasuga, M. (1994) J Biol Chem 269, 31310-31314
Ouali, M., and King, R. D. (2000) Protein Sci 9, 1162-1176
Rahuel, J., Gay, B., Erdmann, D., Strauss, A., Garcia-Echeverria, C., Furet, P., Caravatti, G., Fretz, H., Schoepfer, J., and Grutter, M. G. (1996) Nat Struct Biol 3, 586-589
Rahuel, J., Garcia-Echeverria, C., Furet, P., Strauss, A., Caravatti, G., Fretz, H., Schoepfer, J., and Gay, B. (1998) J Mol Biol 279, 1013-1022
Ren, Z., Cabell, L. A., Schaefer, T. S., and McMurray, J. S. (2003) Bioorg Med Chem Lett 13, 633-636
Rotin, D., Margolis, B., Mohammadi, M., Daly, R. J., Daum, G., Li, N., Fischer, E. H., Burgess, W. H., Ullrich, A., and Schlessinger, J. (1992) Embo J 11, 559-567
Sadowski, I., Stone, J. C., and Pawson, T. (1986) Mol Cell Biol 6, 4396-4408
Schuenke, K. W., Cook, R. G., and Rich, R. R. (1998) Hum Immunol 59, 783-793
Shao, H., Cheng, H. Y., Cook, R. G., and Tweardy, D. J. (2003) Cancer Res 63, 3923-3930
Shao, H., X. Xu, M. A. Mastrangelo, N. Jing, R. G. Cook, G. B. Legge, and D. J. Tweardy. 2004. Structural requirements for signal transducer and activator of transcription 3 binding to phosphotyrosine ligands containing the YXXQ motif. J Biol Chem 279:18967-18973
Shao, H., X. Xu, N. Jing, and D. J. Tweardy. In press. Unique structural determinants for signal transducer and activator of transcription (STAT) 3 recruitment and activation by the granulocyte colony-stimulating factor receptor (G-CSFR) phosphotyrosine ligands 704 and 744. Journal of Immunology
Sheinerman, F. B., Al-Lazikani, B., and Honig, B. (2003) J Mol Biol 334, 823-841
Songyang, Z., Shoelson, S. E., Chaudhuri, M., Gish, G., Pawson, T., Haser, W. G., King, F., Roberts, T., Ratnofsky, S., Lechleider, R. J., and et al. (1993) Cell 72, 767-778
Songyang, Z., Shoelson, S. E., McGlade, J., Olivier, P., Pawson, T., Bustelo, X. R., Barbacid, M., Sabe, H., Hanafusa, H., Yi, T., and et al. (1994) Mol Cell Biol 14, 2777-2785
Stahl, N., Farruggella, T. J., Boulton, T. G., Zhong, Z., Darnell, J. E., Jr., and Yancopoulos, G. D. (1995) Science 267, 1349-1353
Turkson, J., Bowman, T., Garcia, R., Caldenhoven, R., DeGroot, R. P., and Jove, R. (1998) Molecular and Cellular Biology 18, 2545-2552
Turkson, J., Ryan, D., Kim, J. S., Zhang, Y., Chen, Z., Haura, E., Laudano, A., Sebti, S., Hamilton, A. D., and Jove, R. (2001) J Biol Chem 276, 45443-45455
Ullrich, A., Coussens, L., Hayflick, J. S., Dull, T. J., Gray, A., Tam, A. W., Lee, J., Yarden, Y., Libermann, T. A., Schlessinger, J., and et al. (1984) Nature 309, 418-425 Waksman, G., Shoelson, S. E., Pant, N., Cowburn, D., and Kuriyan, J. (1993) Cell 72, 779-790
Weber-Nordt, R. M., Riley, J. K., Greenlund, A. C., Moore, K. W., Darnell, J. E., and Schreiber, R. D. (1996) J Biol Chem 271, 27954-27961
Wegenka, U. M., Buschmann, J., Lutticken, C., Heinrich, P. C., and Horn, F. (1993) Mol Cell Biol 13, 276-288
Wiederkehr-Adam, M., Ernst, P., Muller, K., Bieck, E., Gombert, F. O., Ottl, J., Graff, P., Grossmuller, F., and Heim, M. H. (2003) J Biol Chem 278, 16117-16128
Xia, L., Wang, L., Chung, A. S., Ivanov, S. S., Ling, M. Y., Dragoi, A. M., Platt, A., Gilmer, T. M., Fu, X. Y., and Chin, Y. E. (2002) J Biol Chem
Zhong, Z., Wen, Z., and Darnell, J. E., Jr. (1994) Science 264, 95-98
Ziegler, S F, Davis, T, Schneringer J A et al., New Biologist 3:1242-8, 1991

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

We claim:

1. A Stat3 inhibitor comprising a beta-turn mimetic wherein said beta-turn mimetic is capable of binding to a sequence located within the SH2 domain of Stat3.

2. The Stat3 inhibitor of claim 1, wherein said beta-turn mimetic is a mimetic of a beta-turn region comprising SEQ ID NO:2.

3. The Stat3 inhibitor of claim 1, wherein said beta-turn mimetic comprises a peptide.

4. The Stat3 inhibitor of claim 3, wherein the peptide is amino-terminally modified.

5. The Stat3 inhibitor of claim 3, wherein the peptide is carboxy-terminally modified.

6. The Stat3 inhibitor of claim 3, wherein the peptide comprises a combination of standard amino acids and modified amino acids.

7. The Stat3 inhibitor of claim 3, wherein the peptide comprises the sequence SEQ ID NO:2.

8. The Stat3 inhibitor of claim 3 wherein the peptide comprises the sequence SEQ ID NO:3 (pY1068 dodecapeptide).

9. The Stat3 inhibitor of claim 3 wherein the peptide comprises the sequence SEQ ID NO:4 (pY1086 dodecapeptide).

10. The Stat3 inhibitor of claim 3 wherein the peptide comprises the sequence SEQ ID NO:17 (pY704 dodecapeptide).

11. The Stat3 inhibitor of claim 3 wherein the peptide comprises the sequence SEQ ID NO:19 (pY744 dodecapeptide).

12. The Stat3 inhibitor of claim 2, wherein X2 of SEQ ID NO:2 is not asparagine.

13. The Stat3 inhibitor of claim 1, wherein said mimetic is cyclic.

14. The Stat3 inhibitor of claim 1, wherein said mimetic comprises a peptide having the sequence SEQ ID NO: 23 (X1X2X3Q), wherein X1 is a phosphotyrosine mimetic residue that is selected from the group consisting of phosphonomethylphenylalanine, difluorophosphonomethylphenylalanine, O-malonyltyrosine, and O-fluoromalonyltyrosine.

15. The Stat3 inhibitor of claim 1, wherein binding the sequence within the SH2 domain comprises interaction with residue E638 of Stat3.

16. The Stat3 inhibitor of claim 20, wherein binding the sequence within the SH2 domain further comprises interaction with residues K589, R607, or both.

17. A pharmaceutical composition comprising the Stat3 inhibitor of claim 1.

18. A method of inhibiting Stat3 in at least one cell of an individual, comprising administering to the individual a Stat3 inhibitor of claim 1.

19. A method of treating cancer in an individual, comprising administering to the individual a Stat3 inhibitor of claim 1.

20. The method of claim 19, wherein the cancer is selected from the group consisting of head and neck, breast, prostate, renal cell, melanoma, ovarian, lung, leukemia, lymphoma, and multiple myeloma.

21. The method of claim 19, wherein the cancer is further defined as chemotherapy-resistant cancer.

22. A method of inhibiting Stat3 in at least one cell of an individual, comprising administering to the individual a composition of FIG. 10, FIG. 11, or a mixture thereof.

23. A method of treating cancer in an individual, comprising administering to the individual a composition of FIG. 10, FIG. 11, or a mixture thereof.

24. The method of claim 23, wherein the cancer is selected from the group consisting of head and neck, breast, prostate, renal cell, melanoma, ovarian, lung, leukemia, lymphoma, and multiple myeloma.

25. The method of claim 23, wherein the cancer is further defined as chemotherapy-resistant cancer.

26. A composition dispersed in a pharmaceutically acceptable carrier and comprising a a composition of FIG. 10, FIG. 11, or a mixture thereof.

27. A method of screening for an inhibitor of Stat3, comprising:
providing a Stat3 SH2 domain, wherein said domain is capable of binding to a beta turn in a Stat3-interacting molecule;
providing a test compound; and
assaying binding of said test compound to said SH2 domain, wherein when said test compound binds said SH2 domain, said test compound is said inhibitor.

28. The method of claim 27, further comprising manufacturing the inhibitor.

29. The method of claim 27, wherein said inhibitor is administered to an individual.
